(19) United States
(12) Patent Application Publication
(10) Pub. No.: US 2025/0134659 A1
(43) Pub. Date: May 1, 2025

(54) SYSTEMS AND METHODS FOR TREATING CALCIFIED HEART VALVES

(71) Applicant: EDWARDS LIFESCIENCES CORPORATION, Irvine, CA (US)

(72) Inventors: Brandon Sina Dehdashtian, Irvine, CA (US); Cooper Ryan Rickerson, Huntington Beach, CA (US); Hiroshi Okabe, Tustin, CA (US); Jonathan Yeh, Brea, CA (US); Tejler Javier Gasga, Irvine, CA (US); Madison Paige Kukura, Costa Mesa, CA (US); Zachary Charles VanEvery, Irvine, CA (US); Rani Abdullah Mahmoudi, Huntington Beach, CA (US); Kevin M. Stewart, Powell, TN (US)

(21) Appl. No.: 19/011,433

(22) Filed: Jan. 6, 2025

Related U.S. Application Data
(63) Continuation of application No. PCT/US2023/027072, filed on Jul. 7, 2023.
(60) Provisional application No. 63/359,686, filed on Jul. 8, 2022.

(51) Int. Cl. A61F 2/24; A61B 17/3201

(52) U.S. Cl. CPC A61F 2/2427; A61B 17/3201; A61F 2/2418; A61F 2210/0014; A61F 2220/0008; A61F 2250/0003

(57) ABSTRACT

Apparatuses, systems, and methods for prosthetic valves. An implantation site may comprise a native heart valve or another implantation site in examples. Examples may be utilized for improved anchoring and sealing of flow (e.g., paravalvular leakage) with a native heart valve having calcification. Examples may include breaking up calcification of a native heart valve to improve deployment to the native heart valve.

### CROSS REFERENCE TO RELATED APPLICATIONS

This application is a continuation of International Application No. PCT/US2023/027072, filed Jul. 7, 2023, which designates the United States and was published in English by the International Bureau on Jan. 11, 2024 as WO2024/010892, which claims the benefit of U.S. Provisional Application No. 63/359,686, filed Jul. 8, 2022, the entire contents of each of which are hereby incorporated by reference.

### BACKGROUND

### Field

Certain features of the disclosure relate generally to implants, including prosthetic valves for deployment.

### Background

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures.

These replacement valves are often intended to allow fluid flow therethrough, while sealing or blocking fluid flow on the outside of the replacement valve (paravalvular leakage (PVL)). Such sealing issues may be increased if the native heart valve has an irregular shape or has calcification that may reduce the possibility of proper sealing around the replacement valve. An irregular shape or calcification may also reduce the possibility of proper anchoring of the replacement valve to the native heart valve.

### SUMMARY

Examples of prosthetic valves disclosed herein may be directed to improvements in prosthetic valves. Such prosthetic valves may comprise replacement heart valves in examples. Examples may be utilized for improved anchoring and sealing of flow (e.g., paravalvular leakage) with a native heart valve having calcification. Examples may reduce the possibility of obstruction of a left ventricular outflow tract (LVOT) of a heart, whether resulting from implantation of a prosthetic valve or otherwise. Various other improvements are disclosed.

Examples disclosed herein may include a system for a heart. The system may include a prosthetic valve configured to be deployed to a native valve of a heart and configured to transmit shock waves to break up calcification of the native valve.

Examples disclosed herein may include a system for implanting a prosthetic heart valve in a calcified native valve. The system may include the prosthetic heart valve including: a support structure having an inlet end portion and an outlet end portion and a passageway, and a valve portion positioned within the passageway of the support structure, wherein the valve portion comprises a plurality of leaflets made from pericardium, wherein the valve portion permits flow of blood through the passageway in one direction for replacing the function of a native heart valve; and a delivery catheter for delivering the prosthetic heart valve to the calcified native valve, the delivery catheter including an actuation mechanism for causing the support structure to vibrate, thereby reducing calcification of the calcified heart valve.

Examples disclosed herein may include a delivery system for a heart. The delivery system may include a first inflatable body configured to expand a prosthetic valve positioned upon the first inflatable body to deploy the prosthetic valve to a native valve; and a second inflatable body surrounding the first inflatable body and configured to transmit shock waves to break up calcification of the native valve.

Examples disclosed herein may include a system for implanting a prosthetic heart valve in a calcified native valve. The system may include the prosthetic heart valve including: a support structure having an inlet end portion and an outlet end portion and a passageway, and a valve portion positioned within the passageway of the support structure, wherein the valve portion comprises a plurality of leaflets made from pericardium, wherein the valve portion permits flow of blood through the passageway in one direction for replacing the function of a native heart valve. The system may include a delivery catheter for the prosthetic heart valve, the delivery catheter including: an elongate shaft, and a first inflatable body coupled to the elongate shaft and adapted to expand the prosthetic heart valve when the prosthetic heart valve is positioned upon the first inflatable body to deploy the prosthetic heart valve to the native valve, a second inflatable body coupled to the elongate shaft and adapted to transmit vibrations to break up calcification of the calcified native valve, and an actuator for producing the vibrations of the second inflatable body.

Examples disclosed herein may include a method comprising reducing calcification of a native heart valve; and deploying a prosthetic heart valve to the native heart valve.

Examples disclosed herein may include a method comprising determining a configuration of an implantation site at a native heart valve having calcification, the implantation site being for a prosthetic heart valve; and selecting a configuration of a distal anchor of the prosthetic heart valve based on the determined configuration of the implantation site.

Examples disclosed herein may include a prosthetic valve configured to be deployed to a native valve of a heart. The prosthetic valve may include one or more prosthetic valve leaflets configured to be positioned in a flow channel; a valve body configured to support the one or more prosthetic valve leaflets; and at least one anchor coupled to the valve body and including one or more of a barb or a pad configured to engage calcification of the native valve to anchor to the calcification.

Examples disclosed herein may include a method comprising deploying a prosthetic valve to a native valve. The prosthetic valve may include one or more prosthetic valve leaflets configured to be positioned in a flow channel, a valve body configured to support the one or more prosthetic valve leaflets, and at least one anchor coupled to the valve body and including one or more of a barb or a pad configured to engage calcification of the native valve to anchor to the calcification.

Examples disclosed herein may include a prosthetic valve configured to be deployed to a native valve of a heart. The prosthetic valve may include one or more prosthetic valve leaflets configured to be positioned in a flow channel; and a valve body configured to support the one or more prosthetic valve leaflets and including an outer surface comprising a conformable anchoring surface configured to conform to a shape of calcification of the native valve to anchor to the native valve.

Examples disclosed herein may include a method comprising deploying a prosthetic valve to a native valve. The prosthetic valve may include one or more prosthetic valve leaflets configured to be positioned in a flow channel, and a valve body configured to support the one or more prosthetic valve leaflets and including an outer surface comprising a conformable anchoring surface configured to conform to a shape of calcification of the native valve to anchor to the native valve.

Examples disclosed herein may include a prosthetic valve configured to be deployed to a native valve of a heart. The prosthetic valve may include one or more prosthetic valve leaflets configured to be positioned in a flow channel; and a valve body configured to support the one or more prosthetic valve leaflets, the valve body including an atrial anchor comprising a flange configured to extend radially outward from the flow channel.

Examples disclosed herein may include a prosthetic heart valve configured to be deployed to a native heart valve. The prosthetic heart valve may include a support structure having an inlet end portion and an outlet end portion and a passageway, wherein the support structure includes an atrial anchor comprising a flange for extending radially outward from the passageway. The prosthetic heart valve may include a valve portion positioned within the passageway of the support structure, wherein the valve portion comprises a plurality of leaflets made from pericardium, wherein the valve portion permits flow of blood through the passageway in one direction for replacing the function of the native heart valve.

Examples disclosed herein may include a method comprising deploying a prosthetic valve to a native valve. The prosthetic valve may include one or more prosthetic valve leaflets configured to be positioned in a flow channel, and a valve body configured to support the one or more prosthetic valve leaflets, the valve body including an atrial anchor comprising a flange configured to extend radially outward from the flow channel.

Examples disclosed herein may include a prosthetic valve configured to be deployed to a native valve of a heart. The prosthetic valve may include a valve body; and a spiral body coupled to the valve body and configured to move between an opened state and a closed state to control fluid flow through the valve body.

Examples disclosed herein may include a method comprising deploying a prosthetic valve to a native valve. The prosthetic valve may comprise a valve body, and a spiral body coupled to the valve body and configured to move between an opened state and a closed state to control fluid flow through the valve body.

Examples disclosed herein may include a prosthetic heart valve configured to be deployed to a native heart valve. The prosthetic heart valve may include a support structure having a passageway; and a spiral body coupled to the support structure and positioned within the passageway, the spiral body adapted to move between an opened state and a closed state to control blood flow through the support structure.

Examples disclosed herein may include a system for a heart. The system may comprise a prosthetic heart valve configured to be deployed in a mitral valve of the heart; and an anchor coupled to the prosthetic heart valve and configured to be deployed in a left atrial appendage of the heart.

Examples disclosed herein may include a prosthetic mitral heart valve system for a heart. The system may include a prosthetic mitral heart valve including: a support structure having an inlet end portion and an outlet end portion and a passageway, and a valve portion positioned within the passageway of the support structure, wherein the valve portion comprises a plurality of leaflets made from pericardium, wherein the valve portion permits flow of blood through the passageway in one direction for replacing the function of a native mitral heart valve. The system may include an anchor for deployment in a left atrial appendage of the heart, wherein the anchor is coupled to the prosthetic mitral heart valve for anchoring the prosthetic mitral heart valve within the native mitral heart valve.

Examples disclosed herein may include a method comprising deploying a prosthetic heart valve to a mitral valve of a heart; and deploying an anchor for the prosthetic heart valve to a left atrial appendage of the heart.

Examples disclosed herein may include a method comprising implanting a prosthetic valve at a pulmonary vein of a heart to impede fluid flow to a lung.

Examples disclosed herein may include a stent for a heart. The stent may comprise an outer stent configured to be deployed proximate a left ventricular outflow tract of the heart; and an inner stent positioned within the outer stent and including a flow channel for fluid to pass through the left ventricular outflow tract.

Examples disclosed herein may include a method comprising deploying a stent proximate a left ventricular outflow tract of a heart, the stent including: an outer stent, and an inner stent positioned within the outer stent and including a flow channel for fluid to pass through the left ventricular outflow tract.

Examples disclosed herein may include a system for a heart. The system may comprise a prosthetic heart valve configured to be implanted in a valve of the heart; and an anchor coupled to the prosthetic heart valve and comprising a ventricular chamber configured to extend with a ventricle of the heart.

Examples disclosed herein may include a method comprising deploying a prosthetic heart valve to a valve of a heart; and deploying an anchor for the prosthetic heart valve to a ventricle of the heart, the anchor comprising a ventricular chamber.

Examples disclosed herein may include a method comprising tethering a native mitral heart valve leaflet or removing at least a portion of the native mitral heart valve leaflet to reduce an obstruction by the native mitral heart valve leaflet of a left ventricular outflow tract of a heart.

Examples disclosed herein may include a cutter for at least a portion of a heart valve leaflet, the cutter comprising: a first jaw including a proximal end portion and a distal end portion, the first jaw having a wedge shape converging on an apex at the distal end portion of the first jaw; a second jaw including a proximal end portion and a distal end portion, the second jaw having a wedge shape converging on an apex at the distal end portion of the second jaw; and one or more teeth positioned on one or more of the first jaw or the second jaw and configured to cut the at least the portion of the heart valve leaflet upon the first jaw closing with the second jaw.

Examples disclosed herein may include a prosthetic mitral heart valve system for a heart. The system may include a prosthetic mitral heart valve including: a support structure having an inlet end portion and an outlet end portion and a passageway, and a valve portion positioned within the passageway of the support structure, wherein the valve portion comprises a plurality of leaflets made from pericardium, wherein the valve portion permits flow of blood through the passageway in one direction for replacing the function of a native mitral heart valve. The system may include a tether for tethering a native mitral heart valve leaflet to reduce an obstruction by the native mitral heart valve leaflet of a left ventricular outflow tract of the heart.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1A illustrates an upper perspective view of a prosthetic valve according to examples of the present disclosure.

FIG. 1B illustrates a bottom perspective view of the prosthetic valve shown in FIG. 1A.

FIG. 2 illustrates a side cross sectional schematic view of the prosthetic valve shown in FIG. 1A.

FIG. 3 illustrates a schematic view of a delivery apparatus approaching an implantation site.

FIG. 4 illustrates a side cross sectional schematic view of the prosthetic valve shown in FIG. 1A deployed to a native heart valve.

FIG. 5 illustrates a side cross sectional schematic view of a native heart valve having calcification.

FIG. 6A illustrates a side cross sectional schematic view of a delivery apparatus approaching an implantation site.

FIG. 6B illustrates a side cross sectional schematic view of the delivery apparatus shown in FIG. 6A with an outer inflatable body inflated.

FIG. 6C illustrates a side cross sectional schematic view of the delivery apparatus shown in FIG. 6A with the outer inflatable body inflated.

FIG. 6D illustrates a side cross sectional schematic view of the delivery apparatus shown in FIG. 6A with an implant positioned thereon.

FIG. 6E illustrates a side cross sectional schematic view of the delivery apparatus shown in FIG. 6A with an inner inflatable body inflated.

FIG. 6F illustrates a side cross sectional schematic view of a prosthetic valve deployed to a native heart valve.

FIG. 6G illustrates a side cross sectional view of a delivery apparatus approaching an implantation site.

FIG. 6H illustrates a side cross sectional view of the delivery apparatus shown in FIG. 6G with an inflatable body inflated.

FIG. 6I illustrates a side cross sectional view of the delivery apparatus shown in FIG. 6G advanced from the position shown in FIG. 6H.

FIG. 6J illustrates a side cross sectional view of the delivery apparatus shown in FIG. 6G with an inflatable body inflated.

FIG. 7A illustrates a side view of a delivery apparatus approaching an implantation site.

FIG. 7B illustrates a side view of a prosthetic valve being deployed to an implantation site.

FIG. 7C illustrates a side view of a frame of the prosthetic valve shown in FIG. 7B.

FIG. 7D illustrates a side view of the prosthetic valve shown in FIG. 7B deployed to an implantation site.

FIG. 7E illustrates a side view of the prosthetic valve shown in FIG. 7B deployed to an implantation site.

FIG. 7F illustrates a side cross sectional view of a prosthetic valve including one or more actuators.

FIG. 7G illustrates a side cross sectional view of the prosthetic valve of FIG. 7F deployed to an implantation site.

FIG. 7H illustrates a side cross sectional view of a prosthetic valve including one or more actuators.

FIG. 7I illustrates a side cross sectional view of the prosthetic valve of FIG. 7H deployed to an implantation site.

FIG. 7J illustrates a side cross sectional view of a prosthetic valve with an actuator applied to the prosthetic valve.

FIG. 7K illustrates a side view of the prosthetic valve shown in FIG. 7J deployed to an implantation site.

FIG. 7L illustrates a side cross sectional view of a prosthetic valve with an actuator applied to the prosthetic valve.

FIG. 8 illustrates a side view of calcification of a heart valve being broken up.

FIGS. 9A-9C each illustrate a side view of an anchor.

FIG. 10A illustrates a side cross sectional schematic view of an anchor and calcification.

FIG. 10B illustrates a side cross sectional schematic view of the anchor shown in FIG. 10A anchored to calcification.

FIG. 11A illustrates a side cross sectional schematic view of an anchor and

calcification.

FIG. 11B illustrates a side cross sectional schematic view of the anchor shown in FIG. 11A anchored to calcification.

FIG. 11C illustrates a side cross sectional schematic view of an anchor and calcification.

FIG. 12A illustrates a perspective view of a prosthetic valve.

FIG. 12B illustrates a side cross sectional schematic view of the prosthetic valve shown in FIG. 12A.

FIG. 13 illustrates a side cross sectional schematic view of a prosthetic valve deployed to a native heart valve.

FIG. 14 illustrates a side cross sectional schematic view of a prosthetic valve deployed to a native heart valve.

FIG. 15 illustrates a side cross sectional schematic view of a prosthetic valve deployed to a native heart valve.

FIG. 16A illustrates a perspective view of a prosthetic valve.

FIG. 16B illustrates a side view of the prosthetic valve shown in FIG. 16A deployed to an implantation site and in a closed state.

FIG. 16C illustrates a side view of the prosthetic valve shown in FIG. 16A deployed to an implantation site and in an opened state.

FIG. 17A illustrates a side view of a prosthetic valve deployed to an implantation site and in an opened state.

FIG. 17B illustrates a side view of the prosthetic valve of FIG. 17A in a closed state.

FIG. 18 illustrates a side cross sectional schematic view of a prosthetic valve deployed to a native heart valve and an anchor in a left atrial appendage (LAA).

FIG. 19 illustrates a side cross sectional schematic view of prosthetic valves deployed to pulmonary veins.

FIG. 20 illustrates a side cross sectional schematic view of a prosthetic valve deployed to a native heart valve.

FIG. 21 illustrates a side cross sectional schematic view of a stent deployed proximate a left ventricular outflow tract.

FIG. 22 illustrates a side cross sectional schematic view of a prosthetic valve deployed to an aortic valve and a prosthetic valve deployed to a mitral valve.

FIG. 23A illustrates a side cross sectional schematic view of a cutter approaching a native heart valve leaflet.

FIG. 23B illustrates a perspective view of a cutter approaching a native heart valve leaflet.

FIG. 23C illustrates a perspective view of a cutter cutting a native heart valve leaflet.

FIG. 24A illustrates a side cross sectional view of a cutter approaching a native heart valve leaflet.

FIG. 24B illustrates a view of a surface of a native heart valve leaflet with the cutter applied to the leaflet.

FIG. 24C illustrates a side view of the cutter shown in FIG. 24A with opened jaws.

FIG. 24D illustrates a side view of the cutter shown in FIG. 24A with closed jaws.

FIG. 24E illustrates a view of a surface of a native heart valve leaflet with the cutter having been applied to the leaflet.

FIG. 25A illustrates a side cross sectional schematic view of a cutter approaching a native heart valve leaflet.

FIG. 25B illustrates a side cross sectional schematic view of a cutter approaching a native heart valve leaflet.

FIG. 25C illustrates a side cross sectional schematic view of a snared native heart valve leaflet.

FIG. 26 illustrates a side cross sectional schematic view of a cutter approaching a native heart valve leaflet.

FIG. 27 illustrates a side cross sectional schematic view of a cutter approaching chordae.

FIG. 28A illustrates a heart valve leaflet anchored to a ventricular wall.

FIG. 28B illustrates the heart valve leaflet shown in FIG. 28A anchored to a ventricular wall with a prosthetic valve deployed to a native valve.

### DETAILED DESCRIPTION

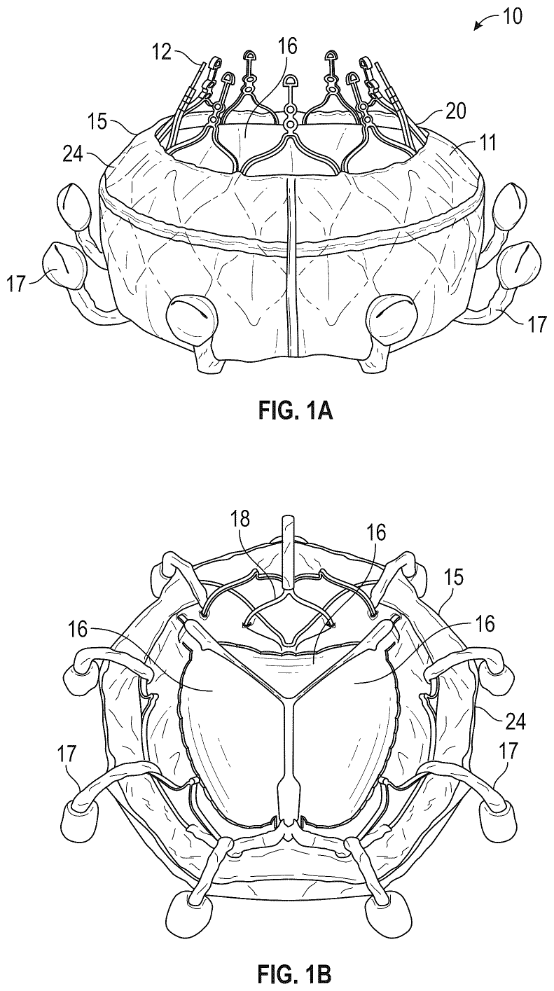

FIG. 1A illustrates a perspective view of a prosthetic valve 10 in the form of a replacement heart valve or prosthetic heart valve. The prosthetic valve 10 may be configured to be deployed within a portion of a patient's body. The prosthetic valve 10, for example, may be deployed to an annulus of a native valve, which may comprise a native mitral valve or a native tricuspid valve. In examples, other implantation locations may be utilized such as within an aortic or pulmonary valve, or in other valves or locations within a patient's body as desired.

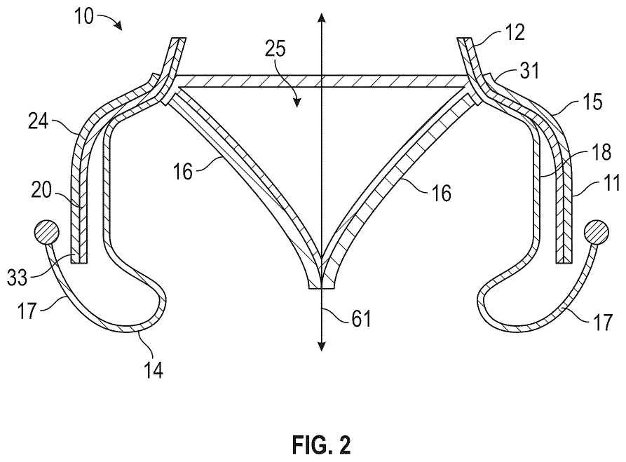

The prosthetic valve 10 may include a proximal end 12 or inlet end portion and a distal end 14 or outlet end portion (marked in FIG. 2), and a length therebetween. The prosthetic valve 10 may further include a valve portion having one or more prosthetic valve leaflets 16, or a plurality of prosthetic valve leaflets 16, configured to be positioned in a flow channel or passageway for controlling flow through the valve 10. The flow channel or passageway may be provided by a support structure 15 of the valve 10. The support structure 15 may form the proximal end 12 or inlet end portion of the prosthetic valve 10 and the distal end 14 or outlet end portion of the prosthetic valve 10. The prosthetic valve leaflets 16 may be configured to move between opened and closed states to mimic and replace the operation of native valve leaflets. The valve portion may be positioned within the passageway of the support structure 15 and may permit flow of blood through the passageway in one direction for replacing the function of a native heart valve. The prosthetic valve leaflets 16 may be made of pericardium or another material as desired.

In examples, the prosthetic valve leaflets 16 may be coupled to a valve body or support structure 15 that may be configured to surround and support the valve portion and the one or more prosthetic valve leaflets 16. The support structure 15 may include a stent or a frame or a support frame (e.g., a valve frame or inner frame or inner support stent 18 and an outer frame or outer support stent 20, among other forms of frames) and a sealing body 11. A valve frame or inner frame or inner support stent 18 is shown in FIGS. 1B and 1n the cross-sectional view of FIG. 2. An outer frame or outer support stent 20 is shown in FIGS. 1A and 2. The outer frame or outer support stent 20 may be part of the sealing body 11 and may be spaced from the inner support stent 18. The outer support stent 20 may surround the inner support stent 18.

Referring to FIGS. 1A and 2, the prosthetic valve 10 may include one or more anchors 17 that may be coupled to the prosthetic valve leaflets 16. The anchors 17 may each be configured to anchor the prosthetic valve leaflets 16 to a portion of a patient's heart, which may comprise a native valve. The anchors 17 may particularly be configured to anchor to the native valve leaflets of the patient's heart. The anchors 17 may extend around the native valve leaflets to anchor to the native valve leaflets. The anchors 17 may comprise distal anchors positioned at the distal end 14 or outlet end portion of the valve 10, or in examples may be positioned in another position as desired.

Each anchor 17 may be configured as a protruding arm configured to extend distally and then curve in a proximal direction to the tip of the respective one of the anchors 17. Such a configuration may allow the anchor 17 to extend around a native leaflet and around the distal tip of the leaflet, to hook over the distal tip of the native valve leaflet and be positioned radially outward of an outward facing surface of a leaflet of the native valve. The anchors 17 may be configured to be in a hooked configuration as shown in FIGS. 1A-2 for example. The anchor 17 may thus resist a force applied in the atrial or proximal direction to the valve 10 and may anchor the valve 10 within the native valve annulus. Other configurations of anchors 17 may be utilized in examples as desired.

The prosthetic valve leaflets 16 may surround a passageway or flow channel 25 as marked in FIG. 2 and may move between open and closed states to control flow through the passageway or flow channel 25. As shown in FIG. 2, the proximal end of the prosthetic valve 10 may comprise an inflow end of the valve 10, and the distal end of the prosthetic valve 10 may comprise an outflow end, although other configurations may be utilized as desired. The prosthetic valve leaflets 16 may be positioned around a central axis 61 of the prosthetic valve 10. The inner support stent 18 and outer support stent 20 may each surround the central axis 61 of the prosthetic valve 10.

Referring again to FIG. 1A, the prosthetic valve 10 may include a sealing body 11. The scaling body 11 may be positioned radially outward from the prosthetic valve leaflets 16 and may be configured to seal against a portion of the native valve. The sealing body 11 may comprise the outer surface of the valve 10. The sealing body 11 may define the outer diameter of the valve 10 and may comprise the outer periphery of the valve 10. The sealing body 11 may include a proximal portion having a proximal end 31 and may include a distal portion having a distal end 33 (marked in FIG. 2).

Referring to the cross-sectional view of FIG. 2, the sealing body 11 may include the frame or outer frame or outer support stent 20 and a sealing skirt 24, or in examples may comprise only a frame or only a sealing skirt as desired. The outer support stent 20 may be positioned radially outward from the inner frame or inner support stent 18. The sealing skirt 24 may be coupled to the outer support stent 20 and may comprise the outer portion of the sealing body 11 as shown in FIG. 1A.

The sealing skirt 24 may be made of a material that resists fluid flow therethrough, such as a cloth material, woven material, or other material such as a polymer or other material that resists fluid flow therethrough. The material may comprise a fabric. A variety of materials may be utilized for the skirt 24 as desired.

The sealing body 11 may be configured to abut a portion of the patient's heart to reduce fluid flow. The skirt 24 may be configured to seal a portion of the native valve annulus. For example, the sealing body 11 may abut a surface of a patient's native valve leaflet to reduce fluid flow between the sealing body 11 and the native leaflet. The sealing body 11 may be configured to abut other portions of the patient's heart to reduce fluid flow as desired.

In examples, the sealing body 11 may be flexible to allow for movement and conformability to a native valve annulus.

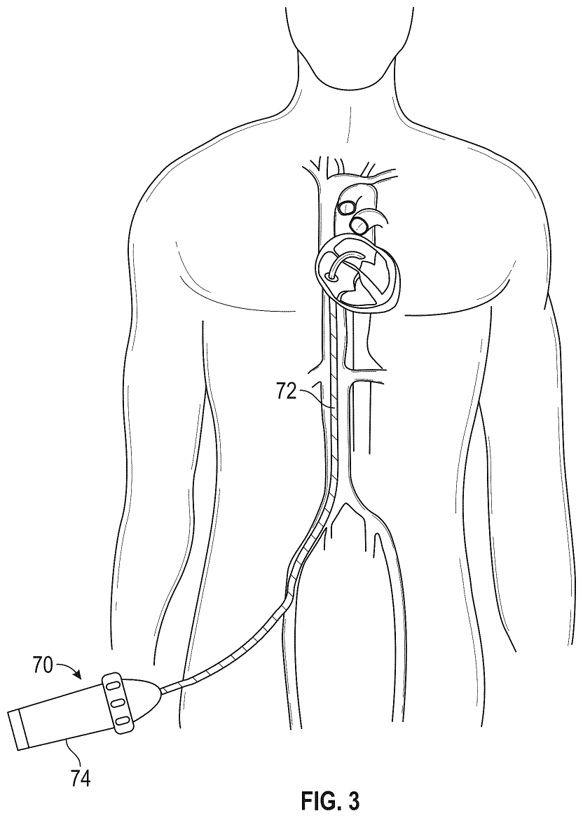

FIG. 3 illustrates advancement of a delivery system 70 or delivery catheter for deployment of the prosthetic valve 10 to an implantation site. The delivery system 70 may include an elongate shaft 72 having a proximal portion and a distal portion, with the proximal portion coupled to a housing in the form of a handle 74. The delivery system 70 may be advanced through the vasculature of a patient, which may include the femoral vein as shown in FIG. 3. Other entries may be utilized in examples, including transapical, or via surgical methods such as thoracotomy or open heart surgery.

In examples, the prosthetic valve 10 may be positioned within an implant retention area of the delivery system 70 and may be covered with a capsule or may otherwise be retained prior to deployment. The prosthetic valve 10 may be deployed as a self-expanding prosthetic. A self-expanding prosthetic may be made of a shape memory material. The shape memory material may comprise nitinol or may have other forms in examples (such as other forms disclosed herein). In examples, however, the prosthetic valve may be a balloon-expandable prosthetic (e.g., positioned upon an inflatable body or balloon upon entry into the patient's body, or slid onto an inflatable body or balloon within the patient's body), or may be mechanically expanded, among other forms of deployment.

The delivery system 70 may be advanced to pass into an atrium of a heart and may pass transeptally into another atrium (e.g., from the right atrium to the left atrium) to reach an implantation site. Such a delivery approach may be utilized for mitral native valve access for example. In examples, the delivery system 70 may extend to the right atrium for tricuspid access, or other delivery approaches to other implantation sites may be utilized in examples as desired.

The prosthetic valve 10 may be held in a compressed configuration within a capsule of the delivery system 70. The anchors 17 may be advanced and may deploy radially outward from the capsule.

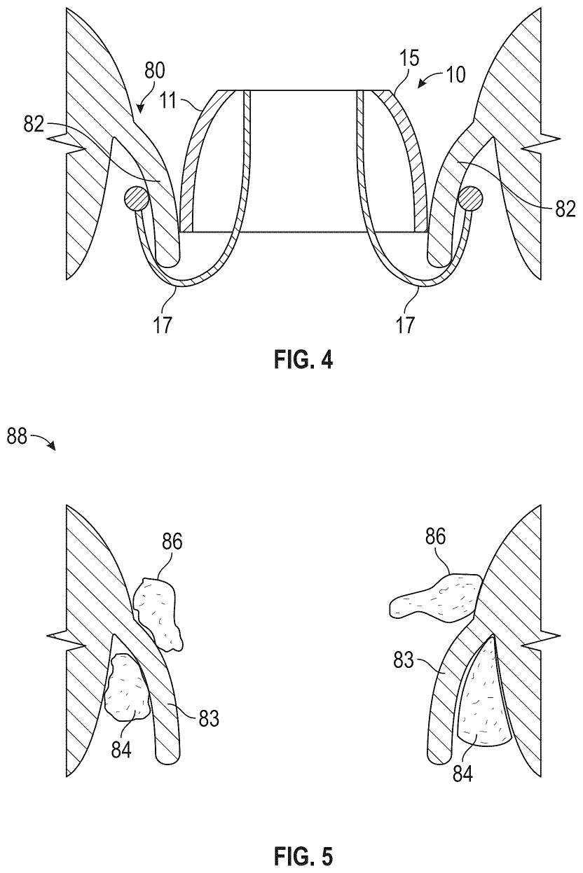

FIG. 4 illustrates the prosthetic valve 10 deployed to the native valve 80 (e.g., a native mitral valve). The sealing body 11 may extend radially outward to contact the inward facing surfaces of the native valve leaflets 82. The anchors 17 may hook over native valve leaflets 82 such that the tips of the anchors 17 are positioned radially outward of the native valve leaflets 82.

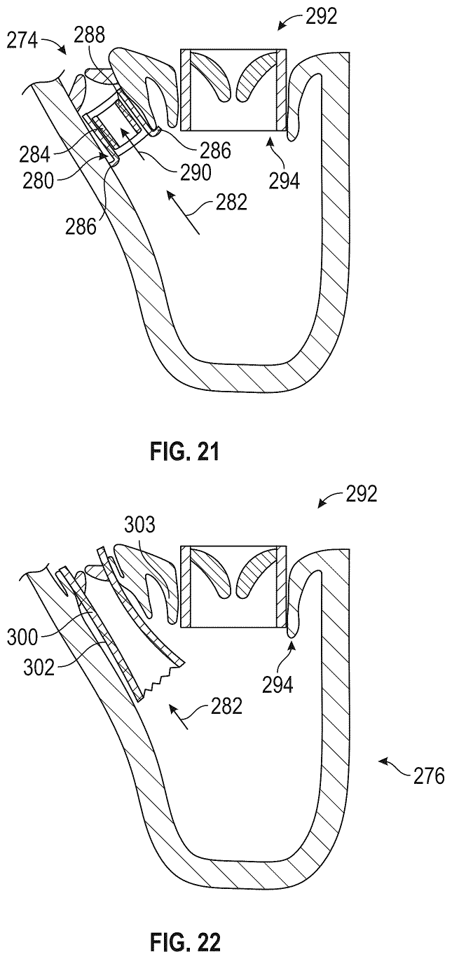

In examples, a configuration of the native valve 80 that includes calcification may impede the ability of a prosthetic valve to properly deploy to an implantation site. For example, calcification may impede the ability of a prosthetic valve to seal with a native valve. Calcification may impede the ability of a prosthetic valve to anchor to a native valve. The native valve 80 may comprise a calcified native mitral valve for example. Calcification may result in an obstruction of the left ventricular outflow tract (LVOT) 282 as marked in FIG. 21, for example, based on implantation of a prosthetic valve to a native mitral valve having calcification. Calcification may produce other undesired effects for the implantation of a prosthetic valve or other treatment of a native valve or a heart.

For example, referring to FIG. 5, calcification 84 may be present underneath or radially outward of an outward facing surface of a leaflet 83 of a native heart valve 88 (e.g., a calcified native mitral valve). Such calcification 84 may impede the ability of anchors 17 as shown in FIG. 4 for example, from hooking around the native valve leaflets 83 to anchor to the native valve leaflets 83. The calcification 84 may block the anchors 17 from being positioned radially outward of the outward surface of the leaflet 83 in a desired manner.

Calcification 86 may be positioned radially inward of a leaflet 83. For example, calcification 86 may be positioned on an inward facing surface of a leaflet 83 or may protrude inward towards the flow channel between the leaflets 83. In examples, calcification 86 may be positioned on the annulus of the native heart valve 88 or may be positioned within the atrium of the heart. Such calcification 86 may impede the ability of a prosthetic valve from scaling or anchoring to the native valve. An irregular shaped annulus may result, which may impede the ability of a prosthetic valve to deploy to the native heart valve 88 in a desired manner. The calcification may be at the mitral valve, and may comprise mitral annular calcification (MAC).

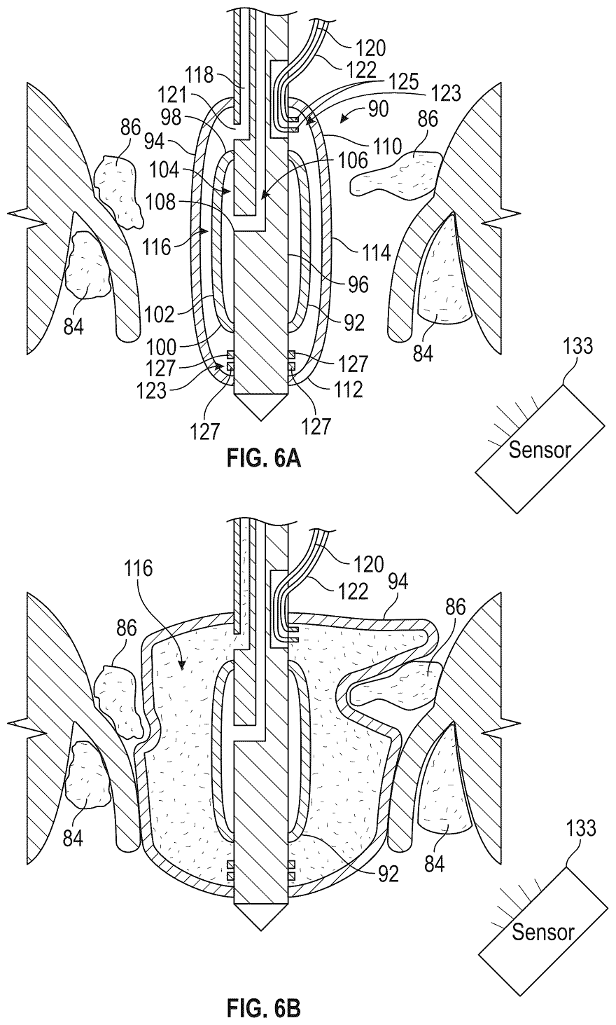

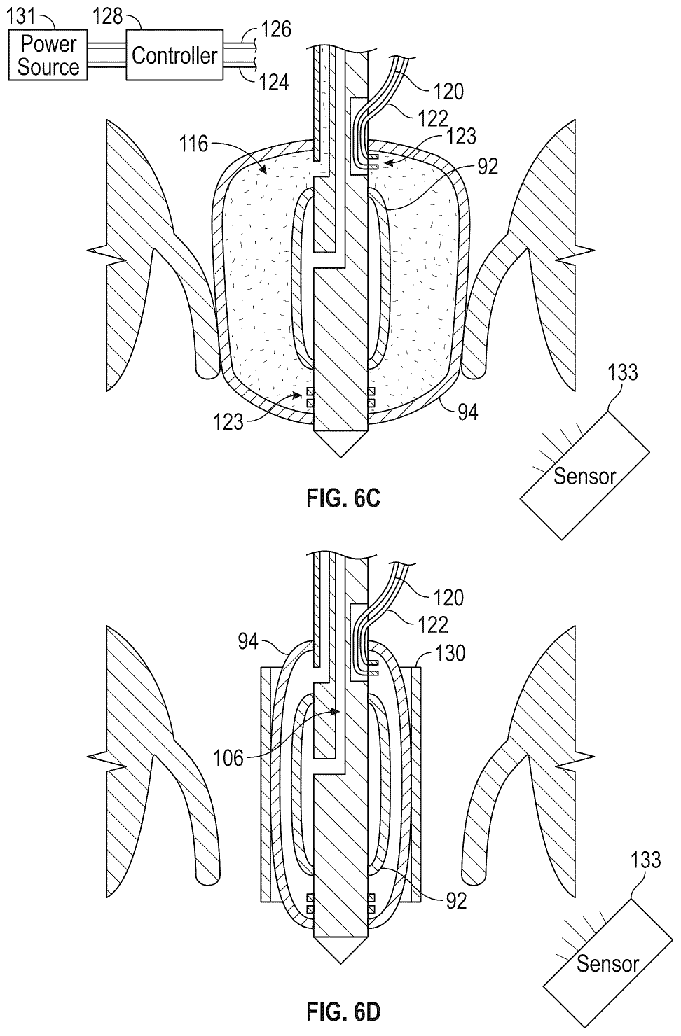

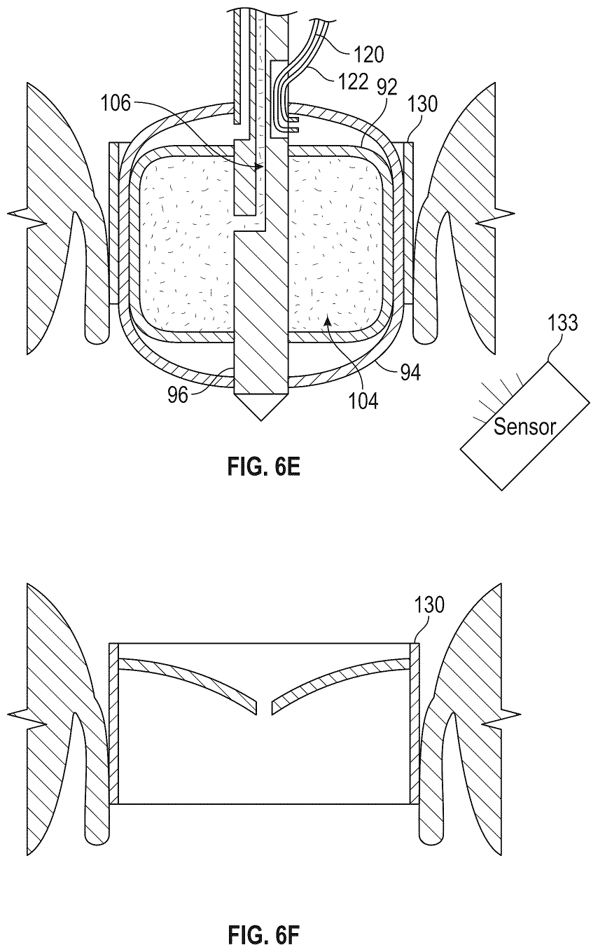

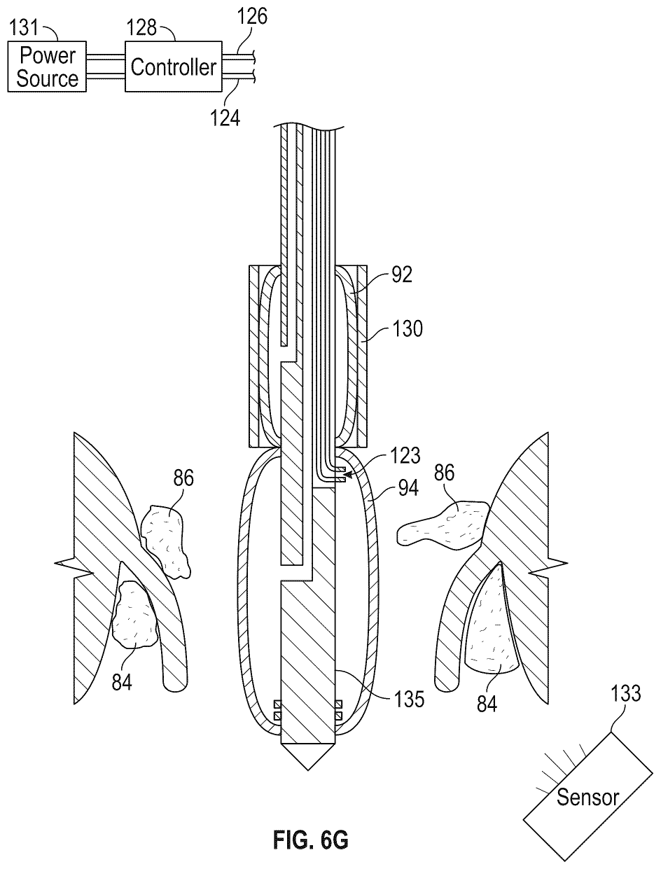

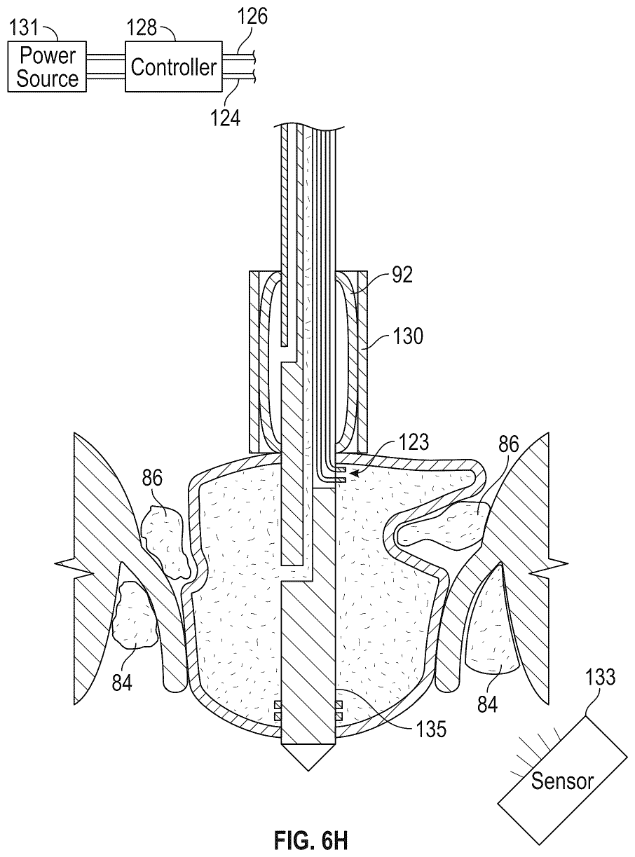

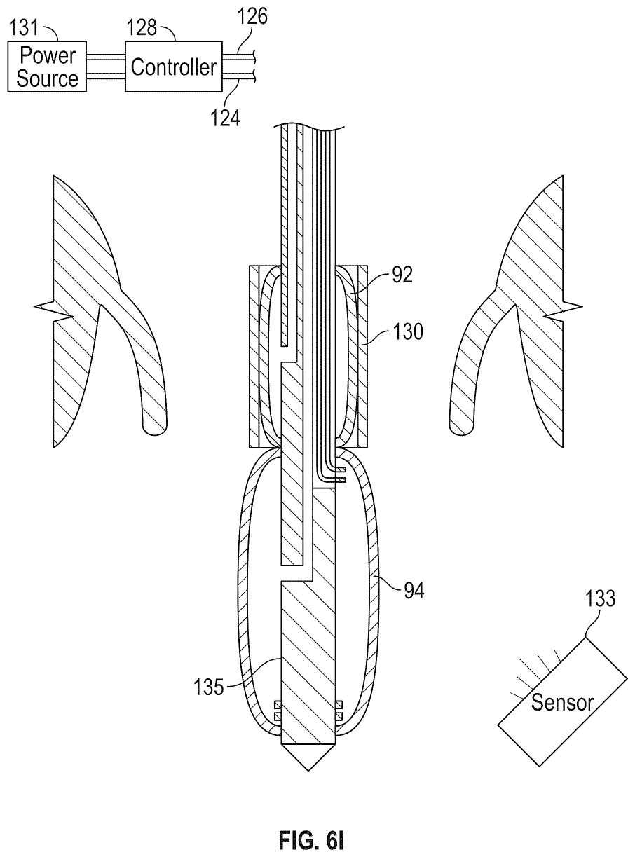

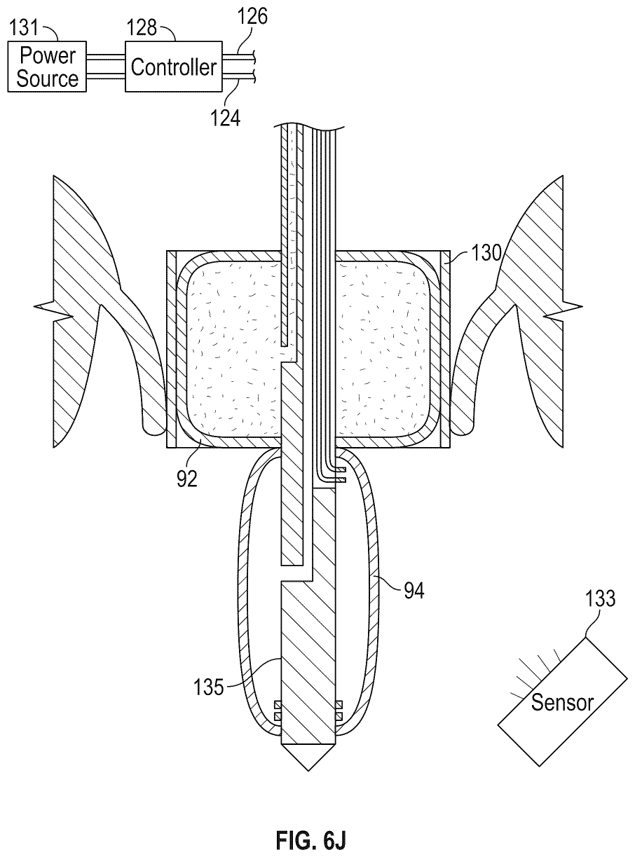

In examples, calcification may be beneficially reduced to reduce the possibility of adverse effects upon deployment of a prosthetic valve, or adverse effects upon the operation of a heart generally. FIGS. 6A-6F illustrate an example of a delivery system 90 or delivery catheter that may be utilized in examples herein.

The delivery system 90 or delivery catheter may include a first inflatable body 92 and a second inflatable body 94. The first inflatable body 92 may be configured to expand a prosthetic valve when the prosthetic valve is positioned upon the first inflatable body 92 to deploy the prosthetic valve to a native valve. The second inflatable body 94 may surround the first inflatable body 92 and may be configured to transmit vibrations to break up calcification of the native valve. The delivery system 90 or delivery catheter may be for deployment of a prosthetic valve.

The first inflatable body 92 and the second inflatable body 94 may each be coupled to an elongate shaft 96 of a delivery apparatus or delivery catheter, similar to the elongate shaft 72 shown in FIG. 3.

A distal end portion of the elongate shaft 96 may be shown in FIG. 6A and a proximal end portion of the elongate shaft 96 may be coupled to a handle similar to the handle 74 shown in FIG. 3. The handle may include a control mechanism (as represented with the control knob shown in FIG. 3) that may be for controlling a deflection of the elongate shaft 96 for navigation through a patient's vasculature. The control mechanism may be configured to position the inflatable bodies 92, 94 in a desired orientation relative to an implantation site. The first inflatable body 92 and the second inflatable body 94 may each be coupled to the distal end portion of the elongate shaft 96.

The first inflatable body 92 may include a proximal end portion 98 and a distal end portion 100 and a central portion 102. The proximal end portion 98 may be coupled to the elongate shaft 96 and the distal end portion 100 may be coupled to the elongate shaft 96. The central portion 102 may bound a cavity 104 that may be configured to receive fluid for inflating the first inflatable body 92. The cavity 104 may be in fluid communication with a first fluid conduit 106 that may extend along the elongate shaft 96. A port 108 may allow for fluid transfer between the first fluid conduit 106 and the cavity 104. The first fluid conduit 106 may comprise a lumen of the elongate shaft 96 in examples. The first fluid conduit 106 may receive fluid from a reservoir that passes fluid along the lumen of the elongate shaft 96 for example.

The second inflatable body 94 may extend over the first inflatable body 92. The first inflatable body 92 may be positioned within the second inflatable body 94. The second inflatable body 94 may include a proximal end portion 110 and a distal end portion 112 and a central portion 114. The proximal end portion 110 may be positioned proximal of the proximal end portion 98 of the first inflatable body 92 and may be coupled to the elongate shaft 96. The distal end portion 112 may be positioned distal of the distal end portion 100 of the first inflatable body 92 and may be coupled to the elongate shaft 96.

The central portion 114 of the second inflatable body 94 may bound a cavity 116 that may be configured to receive fluid for inflating the second inflatable body 94. The cavity 116 may be in fluid communication with a second fluid conduit 118 that may extend along the elongate shaft 96. The second fluid conduit 118 may extend around the first fluid conduit 106, such that the elongate shaft 96 has a double lumen configuration. The second fluid conduit 118 may receive fluid from a reservoir that passes fluid along the lumen of the second fluid conduit 118 for example. Other configurations may be utilized in examples. A port 121 may allow for fluid transfer between the second fluid conduit 118 and the cavity 116. The second fluid conduit 118 may comprise a lumen of the elongate shaft 96 in examples.

The second inflatable body 94 may be configured to inflate independent of the first inflatable body 92. For example, the second fluid conduit 118 may be configured to deliver fluid to inflate the second inflatable body 94 without inflation of the first inflatable body 92. A fluid control device (e.g., a syringe or other form of fluid control device) may be utilized to independently control fluid flow to the first inflatable body 92 and the second inflatable body 94. The fluid control device may provide fluid from one or more reservoirs as desired.

The second inflatable body 94, in examples, may be more compliant than the first inflatable body 92. The second inflatable body 94 may be more compliant to conform to a shape of a native valve that may include calcification. The first inflatable body 92 may be less compliant than the second inflatable body 94 to support a prosthetic valve that may be positioned upon the first inflatable body 92 and expanded by the first inflatable body 92.

In examples, the second inflatable body 94 may be configured to transmit vibrations to break up calcification. The vibrations may comprise acoustic pressure waves in examples. The acoustic pressure waves may comprise ultrasonic waves in examples. The ultrasonic waves may comprise shock waves in examples. Various other forms of vibrations or frequencies of waves may be utilized to produce a desired result in examples (e.g., low frequency waves at less than an ultrasonic frequency may be utilized in examples, and higher frequency waves may be utilized). The vibrations may be configured to break up the calcification into particles in a process similar to lithotripsy. One or more actuators 123, for example, may be utilized to produce the vibrations of the second inflatable body 94.

The actuators 123 may have a variety of forms in examples. The actuators 123, for example, may comprise electrodes 125. The electrodes 125 may be configured to excite or vaporize a fluid filling the second inflatable body 94 to produce the vibrations of the second inflatable body 94. The electrodes 125, for example, may produce an electrical arc or spark upon a voltage being applied to the electrodes 125. The electrical arc or spark may vaporize a part of the fluid filling the second inflatable body 94 (e.g., a portion of the fluid adjacent to the electrodes 125), with the gas pressure of vaporization producing the vibrations of the second inflatable body 94. The vibrations may propagate through the fluid to the outer surface of the second inflatable body 94 and apply the vibrations to the calcification of the native valve. Other forms of actuators or other methods of use of actuators may be utilized in examples.

Electrical conduits 120, 122 may extend to the electrodes 125 for providing the electrical energy to the electrodes 125 that produce the electrical arc or spark. A positive electrical conduit 120 and negative electrical conduit 122 may be provided to produce the resulting electrical arc or spark. The conduits 120, 122 may couple to respective electrodes 125 in examples. The electrical conduits 120, 122 may extend along the elongate shaft 96 and may be adapted to provide electrical energy to the electrodes 125. Similar electrical conduits may extend to other electrodes 127 (marked in FIG. 6A) that may be positioned in other locations within the second inflatable body 94. The electrodes 125, 127 may be configured as rings extending about the elongate shaft 96 or may have other configurations in examples.

Proximal end portions of the electrical conduits 120, 122 may couple to contacts 124, 126 of a controller 128 (marked in FIG. 6C) that may be configured to cause the actuators 123 to transmit the vibrations to the calcification. The controller 128 may receive power from a power source 131 (e.g., a battery or power connector such as a mains connector) for providing power to the actuators 123. For example, the controller 128 may control the amplitude and the frequency of the electrical energy (e.g., current or voltage) applied to the electrodes 125, 127 to produce the vibrations (e.g., shock waves) applied to the calcification and a duration of time that the vibrations are applied to the calcification. The controller 128 may provide electrical energy resulting in an ultrasonic frequency of the vibrations from the second inflatable body 94. The controller 128 may operate on a user input or may operate automatically in examples.

The second inflatable body 94 may be utilized in a process to break up calcification to provide a reduced calcification implantation site for a prosthetic valve. The calcification may be entirely removed from a native heart valve or may be partially removed to provide an improved implantation site for the prosthetic valve. The calcification may be removed from a calcified native mitral valve in examples (or other native valves as desired).

For example, referring to FIG. 6B, the second inflatable body 94 may be positioned at an implantation site and may be expanded. Fluid may fill the cavity 116 to expand the second inflatable body 94. The second inflatable body 94 may conform to the shape of the native valve and to calcification of the native valve (e.g., calcification 86 that may be interior of the native valve leaflets and calcification 84 that may be outward of the native valve leaflets). The first inflatable body 92 may remain uninflated at this point, or may be partially inflated as desired.

Referring to FIG. 6C, the second inflatable body 94 may transmit the vibrations to break up the calcification 86, 84. The actuators 123 may produce the vibrations transmitted through the fluid filling the cavity 116. The controller 128 may control generation of the vibrations of the second inflatable body 94. The vibrations may be applied for a desired duration to fully or partially remove the calcification. In examples, the vibrations may be produced on the surface of the second inflatable body 94.

In examples, a sensor 133 may be provided that may be used to image the reduction of the calcification. The sensor 133, for example, may comprise an imaging sensor (e.g., fluoroscopy or ultrasound or combinations thereof) that images the reduction of the calcification. In examples, the sensor 133 may be configured to image a position of the second inflatable body 94 to determine the reduction of the calcification. The sensor 133 may image a size or diameter of the second inflatable body 94 following or during the calcification reduction process. The second inflatable body 94, for example, may include one or more imaging markers (e.g., a radiopaque material, or echogenic marker) that may be imaged to determine the size or diameter of the second inflatable body 94. A user may determine if sufficient calcification has been reduced based on the output from the sensor 133. In examples, the output from the sensor 133 may be provided as feedback to the controller 128 for the controller 128 to automatically produce the vibrations from the second inflatable body 94 and/or cease producing the vibrations upon a sufficient amount of the calcification being removed. Other configurations may be utilized in examples.

With a desired amount of calcification broken up, a prosthetic valve 130 may be positioned upon the first inflatable body 92 and accordingly upon the second inflatable body 94. The prosthetic valve 130, for example, may comprise a balloon expandable valve and may be slid upon the second inflatable body 94 with the prosthetic valve 130 in an undeployed or unexpanded configuration. The prosthetic valve 130 may include any feature disclosed herein in regard to a prosthetic valve, including any feature of the prosthetic valve 10 or other prosthetic valve disclosed herein. In examples, the prosthetic valve 130 may lack anchors 17 as disclosed in regard to the prosthetic valve 10, or may include such anchors (or other forms of anchors). The prosthetic valve 10 may comprise a single frame prosthetic valve 130 may comprise a multi-frame prosthetic valve as disclosed in regard to the valve 10.

The second inflatable body 94 may be deflated prior to the prosthetic valve 130 being slid onto the second inflatable body 94. The second inflatable body 94 may have an outer surface that is configured to have the prosthetic valve 130 slid onto the outer surface in vivo. FIG. 6D, for example, illustrates a prosthetic valve 130 upon the second inflatable body 94. The second inflatable body 94 is positioned within the flow channel or passageway of the prosthetic valve 130. The first inflatable body 92 may be configured to expand the prosthetic valve 130 positioned upon the first inflatable body 92 and upon the second inflatable body 94. The first inflatable body 92 may then be inflated via the first fluid conduit 106 to expand the prosthetic valve 130 and the second inflatable body 94 and deploy the prosthetic valve 130 to an implantation site.

FIG. 6E, for example, illustrates the first inflatable body 92 having been inflated via the first fluid conduit 106. The first inflatable body 92 may expand radially outward and correspondingly expand the second inflatable body 94 radially outward and the prosthetic valve 130. The prosthetic valve 130 may be deployed in position due to the expansion of the first inflatable body 92. In examples, a sensor 133 may be utilized to image a diameter of the prosthetic valve 130 upon implantation. The sensor 133 may comprise an imaging sensor for imaging the diameter of the prosthetic valve 130. The output from the sensor 133 may be utilized to determine a resulting diameter of the prosthetic valve 130 and whether proper deployment has occurred.

The first inflatable body 92 may be deflated and may be removed from the implantation site along with the second inflatable body 94. The elongate shaft 96 may be withdrawn from the implantation site to remove the first inflatable body 92 and the second inflatable body 94.

FIG. 6F, for example, illustrates the prosthetic valve 130 deployed to an implantation site. The implantation site may have calcification fully or partially removed from the site.

Variations in the systems and methods may be utilized in examples.

FIG. 6G-6J illustrate an example in which the first inflatable body 92 is axially spaced from the second inflatable body 94 along the elongate shaft 135 of the delivery system or delivery catheter. The features of the example of FIGS. 6G-6J may otherwise operate in a similar manner as with the features of FIGS. 6A-6F.

The second inflatable body 94, for example, may be advanced to be positioned adjacent to the calcification 84, 86 of the native valve. Fluid may inflate the second inflatable body 94 to produce an inflated state as shown in FIG. 6H for example. The actuator 123 may be utilized to produce vibrations to break up the calcification in a similar manner as described herein. The sensor 133 may be utilized to image the reduction of the calcification in a similar manner as disclosed herein.

The second inflatable body 94 may be deflated and then the elongate shaft 135 may be advanced distally as represented in FIG. 6I. The first inflatable body 92 may be positioned at the implantation site such that the prosthetic valve 130 is provided in the desired position upon expansion of the first inflatable body 92. The first inflatable body 92 may be inflated (as represented in FIG. 6J) to implant the prosthetic valve 130 at the desired implantation site. A sensor 133 may be utilized to image a diameter of the prosthetic valve 130 upon implantation, as disclosed herein.

The features of FIGS. 6G-6J may beneficially allow for the prosthetic valve 130 to remain upon the first inflatable body 92 during the calcification reduction process of the second inflatable body 94. As such, a prosthetic valve 130 need not be slid onto the first inflatable body 92, yet may remain on the first inflatable body 92 during the calcification reduction process of the second inflatable body 94. In examples, the prosthetic valve 130 may yet be slid onto the first inflatable body 92 during an implantation procedure.

The features of the examples FIGS. 6A-6J may be utilized solely or in combination with the features of any other example herein. Variations in the systems and methods may be utilized in examples.

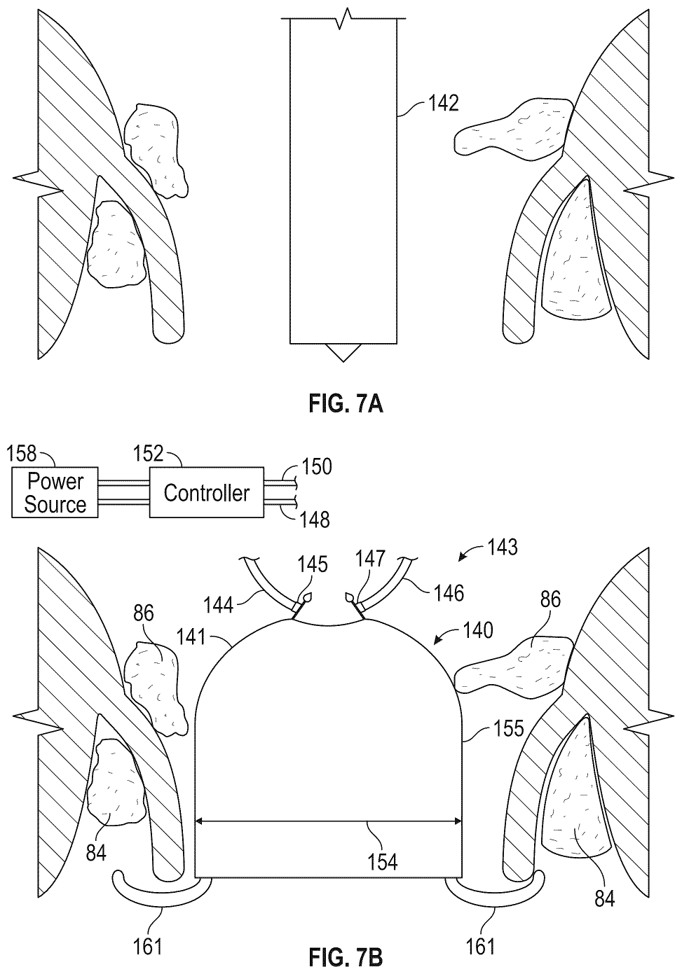

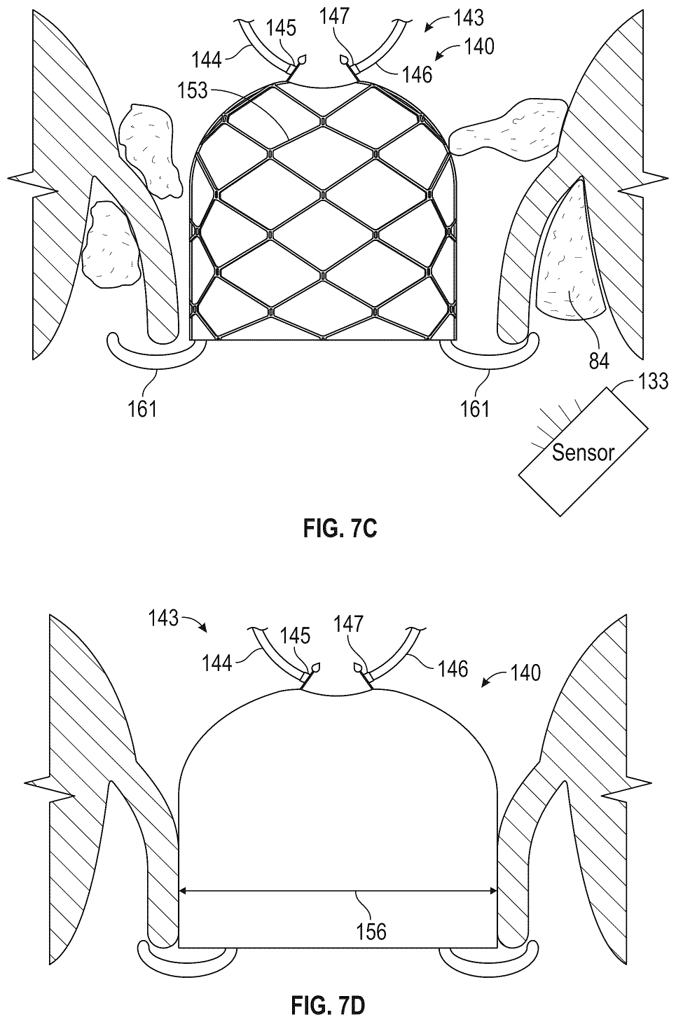

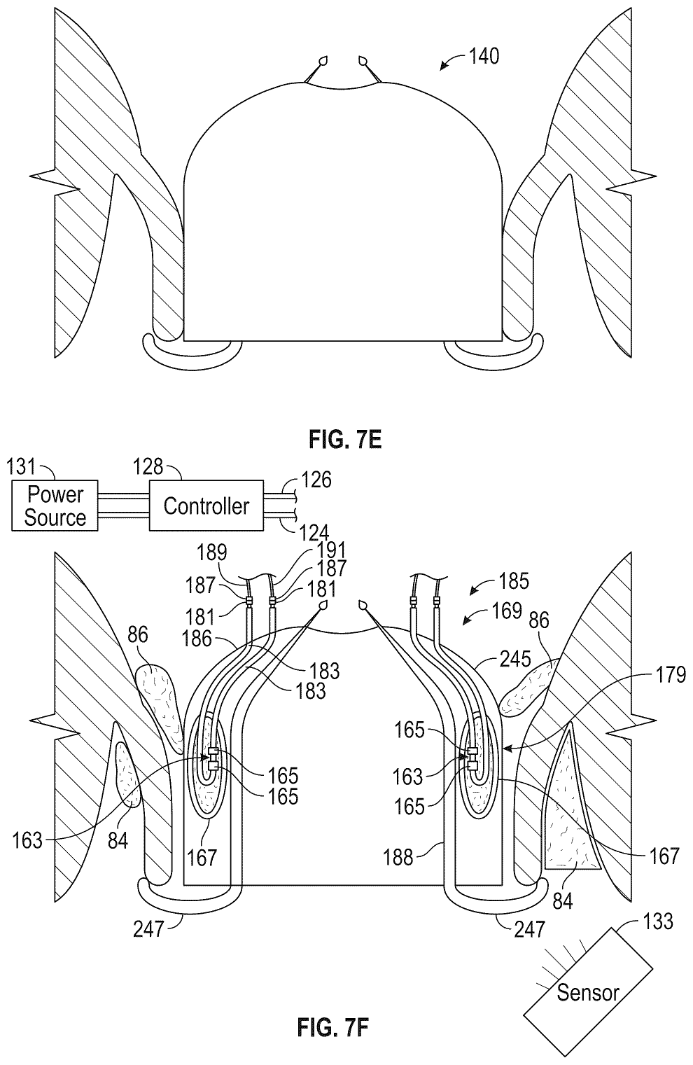

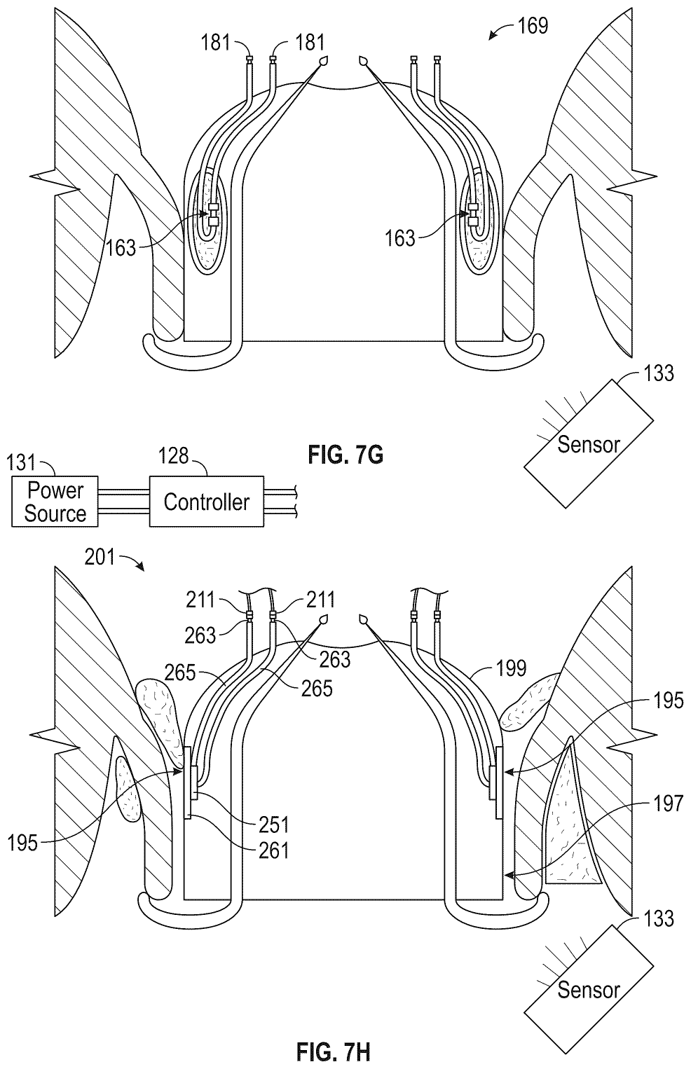

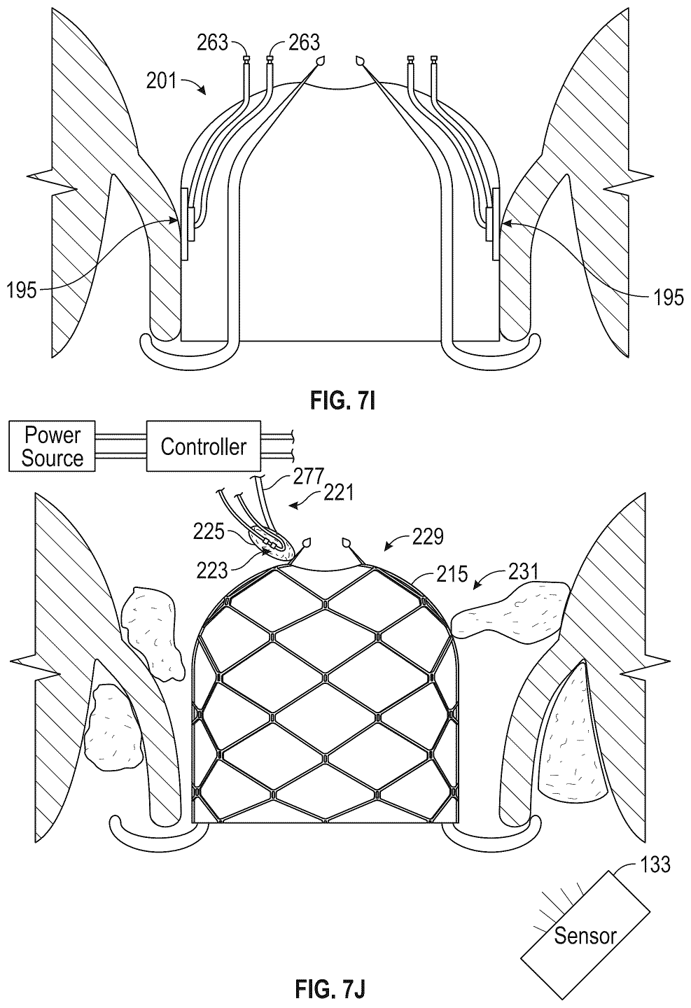

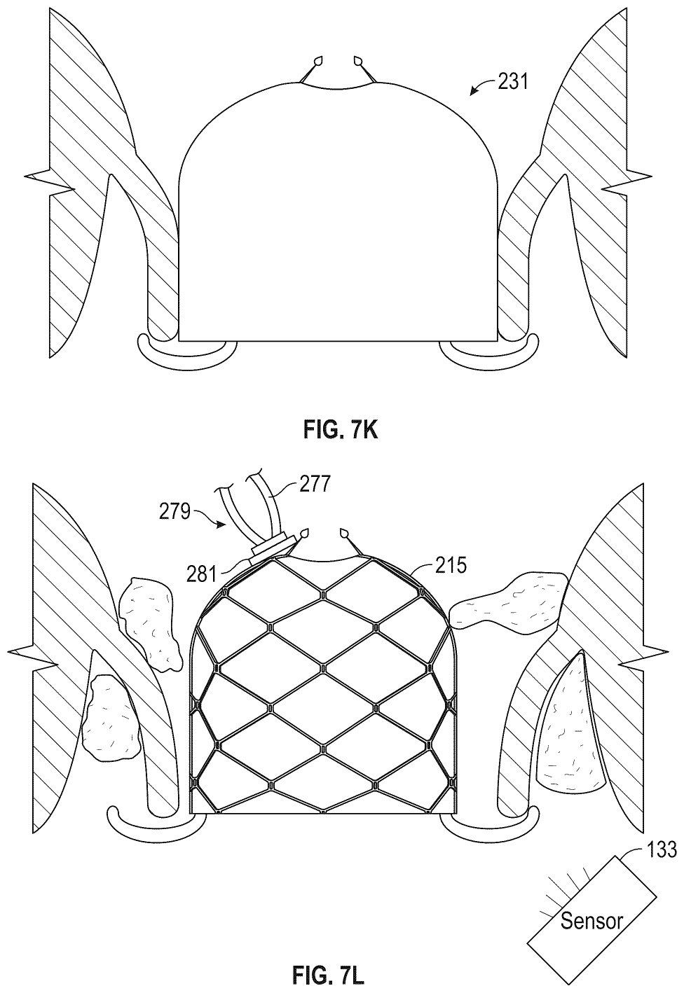

FIGS. 7A-7L illustrate examples in which a prosthetic valve may be configured to deploy to a native valve of a heart and may be configured to transmit vibrations to reduce calcification of the native valve. The vibrations may break up calcification of the native valve. In examples, the prosthetic valve utilized may comprise a self-expanding or self-deploying valve, or may be configured to expand upon a current being applied to a frame of the prosthetic valve. Other forms of prosthetic valves (e.g., balloon expandable or mechanically expandable) may be utilized in examples.

Referring to FIG. 7A, a prosthetic valve 140 may be positioned within a capsule 142 of a delivery apparatus or delivery catheter, such as a delivery apparatus or delivery catheter shown in FIG. 3. The capsule 142 may retain the prosthetic valve therein. Upon the capsule 142 being placed at a desired position relative to an implantation site, the capsule 142 may be retracted to allow the prosthetic valve to deploy. In examples, other forms of retention devices may be utilized to retain the prosthetic valve prior to deployment.

For example, FIG. 7B illustrates the capsule 142 having been retracted. The prosthetic valve 140 may remain undeployed or unexpanded or may initiate a self-expansion upon release from the capsule 142. In examples, the prosthetic valve 140 may transmit vibrations to break up calcification of the native valve.

For example, the delivery apparatus of the delivery catheter may include an actuation mechanism 143 that causes the support structure to vibrate, thereby reducing calcification of the calcified native valve. For example, the prosthetic valve 140 may include a support structure 141. The support structure 141 may be configured similarly as any example of support structure disclosed herein. The support structure 141 may comprise a support frame, which may be configured similarly as any example of frame disclosed herein. A support frame 153 is marked in FIG. 7C for example. The actuation mechanism 143 may be configured to transmit energy to the support structure 141 to cause the support structure 141 to produce the vibrations.

The actuation mechanism 143 may include one or more electric terminals 145, 147 for applying electrical energy to the support structure. The electric terminals 145, 147 may be configured to electrically connect with corresponding terminals on the support structure 141 (or on the support frame 153). The electric terminals 145, 147 may connect with electrical conduits 144, 146 that may be configured to couple to electrical contacts 148, 150 of a controller 152. The electrical conduits 144, 146 may extend along an elongate shaft of the delivery system or delivery catheter. A proximal end portion of the electrical conduits 144, 146 may couple to the electrical contacts 148, 150 of the controller 152.

The controller 152 may receive power from a power source 158 (e.g., a battery or power connector such as a mains connector).

In examples, the controller 152 may be configured to control the vibrations via a current to the support frame 153 that may cause movement of the support frame 153. FIG. 7C, for example, illustrates a support frame 153 of the prosthetic valve 140 (with the sealing body or scaling skirt 155 excluded from view in FIG. 7C). The support frame 153 may be configured to support one or more prosthetic valve leaflets (not shown) in examples. The support frame 153 may surround a passageway or flow channel that the one or more prosthetic valve leaflets are positioned in. In examples, the support frame 153 may support the sealing skirt 155. The support frame 153 may comprise an outer frame or outer support stent or other form of frame in examples. In examples, the support frame 153 may comprise an inner frame or inner support stent or other form of frame in examples. The prosthetic valve 140 may include one or more distal anchors 161 in examples. The distal anchors 161 may be configured to extend over a tip of a leaflet of a native valve or may have another configuration in examples.

The support frame 153, in examples, may comprise a shape memory material that may move in response to a current applied to the support frame 153. The shape memory material may have a variety of forms including a soft superelastic Nitinol, a platinum iridium, or a shape memory Nitinol, among others. The current applied to the support frame 153 may be utilized to produce vibrations from the frame that may be utilized to break up calcification of the native valve.

The controller 152 may control the energy that is transmitted to the support structure 141 to cause the support structure 141 to vibrate. The controller 152, for example, may vary the amount of electric current applied to the support frame 153. The electric current may be provided by the power source 158, which is transmitted to the support frame 153. The controller 152 may control the amplitude and frequency of a current applied to the support frame 153 to produce the vibrations. The controller 152 may apply pulsatile electric current to the frame. The vibrations produced by the prosthetic valve 140 may comprise acoustic pressure waves in examples. The acoustic pressure waves may comprise ultrasonic waves in examples. The ultrasonic waves may comprise shock waves in examples. Various other forms or frequencies of waves may be utilized to produce a desired result in examples (e.g., low frequency waves at less than an ultrasonic frequency may be utilized in examples, and higher frequency waves may be utilized). The vibrations may be produced by the heat provided from the electric current applied to the support frame 153, with resulting vibrations produced due to the movement of the support frame 153. The vibrations may be utilized to break up the calcification 86, 84 shown in FIG. 7B fully or partially. The terminals 145, 147 may couple the controller 152 to the support frame 153 to transmit the electric current to the support frame 153.

In examples, the electric terminals 145, 147 may comprise releasable terminals that may release from the support frame 153 following an implantation procedure. For example, the terminals 145, 147 may be magnetically coupled or coupled with a releasable clip, clamp, or other form of coupler to the support frame 153. As such, the terminals 145, 147 and electrical conduits 144, 146 may be removed following implantation.

The vibrations produced by the prosthetic valve 140 may fully or partially remove the calcification at an implantation site. As such, a reduced calcification may result, which may improve the deployment of the prosthetic valve 140. In examples, a sensor 133 may be utilized that may be utilized to image a diameter of the prosthetic valve 140 upon implantation. The sensor 133 may comprise an imaging sensor for imaging the diameter of the prosthetic valve 140. The output from the sensor 133 may be utilized to determine a resulting diameter of the prosthetic valve 140 and whether proper deployment has occurred. In examples, the output from the sensor 133 may be provided as feedback to the controller 152 of the diameter of the prosthetic valve 140. The feedback may be utilized for the controller 152 to automatically produce the vibrations from the prosthetic valve 140 and/or cease producing the vibrations upon a sufficient amount of the calcification being removed. Other configurations may be utilized in examples.

Referring to FIG. 7D, the prosthetic valve 140 for example, may deploy to the native valve having a reduced calcification. The electric terminals 145, 147 may be removed from the prosthetic valve 140 if desired. A configuration as shown in FIG. 7E, for example, may result.

In examples, the current applied to the frame may be configured to control the expansion of the frame. For example, the frame may be configured to expand in response to current applied to the frame that may produce heat. As shown in FIG. 7B, the prosthetic valve 140 may have a first diameter 154. Current may be applied to the frame to cause the prosthetic valve to expand to a larger second diameter 156 shown in FIG. 7D. The current applied to the support frame 153 via the actuation mechanism 143 may cause the support frame 153 to expand. The electrical energy applied to the support frame 153 may increase the diameter of the support frame 153. In examples, the current may be controlled by the controller 152 to produce a desired expansion of the support frame 153. For example, expansion may be initiated by the controller 152 or may be ceased by the controller 152. A user, for example, may determine that expansion should cease based on a mispositioning of the prosthetic valve 140 or other condition that may require cessation of the expansion. The controller 152 accordingly may cease or reduce current to the frame that may cease expansion. At a desired time, the controller 152 may commence expansion to complete expansion or deployment of the prosthetic valve 140. In examples, the rate of expansion may be controlled by the controller 152. The controller 152 may control the expansion automatically, which may be based on a feedback signal received from sensors or forms of visualization of the deployment. The output from the sensor 133, for example, may be provided as feedback to the controller 152 of the diameter of the prosthetic valve 140.

The controlled expansion of the prosthetic valve 140 may allow the size of the prosthetic valve 140 to be set during expansion. As such, the prosthetic valve 140 may be large enough to reduce the possibility of paravalvular leakage (PVL), yet not be so large as to produce undue force or conduction disturbances upon an implantation site.

In examples, the expansion of the support frame due to current may be utilized solely or in combination with vibrations produced by the prosthetic valve 140. Similarly, the vibrations produced by the prosthetic valve may be utilized solely or in combination with the expansion of the support frame due to current. In examples, the prosthetic heart valve may be expanded in the native heart valve and vibrations may be applied from the prosthetic heart valve to reduce the calcification of the heart valve.

In examples, the support frame may comprise a dock for receiving an insert having prosthetic valve leaflets. In examples, the support frame may be coupled to the prosthetic valve leaflets upon implantation into the patient's body.

Other forms of actuation mechanisms for transmitting energy to a support structure to cause the support structure to produce vibrations for reducing calcification of the calcified native valve may be provided in examples.

FIGS. 7F-7I, for example, illustrate configurations in which the support structure of the prosthetic valve includes one or more actuators for producing the vibrations for reducing the calcification of the calcified native valve. A variety of forms of actuators may be utilized. FIGS. 7F and 7G, for example, illustrate actuators 163 in the form of electrodes 165 that may be configured to excite or vaporize a fluid filling one or more inflatable bodies 167 to produce the vibrations of the inflatable bodies 167 in a similar manner as the actuators 123. For example, the electrodes 165 may produce an electrical arc or spark upon a voltage being applied to the electrodes 165. The electrical arc or spark may vaporize a part of the fluid filling the one or more inflatable bodies 167 (e.g., a portion of the fluid adjacent to the electrodes 165), with the gas pressure of vaporization producing the vibrations of the inflatable bodies 167. The vibrations may propagate through the fluid to the outer surface of the inflatable bodies 167 and apply the vibrations to the calcification of the native valve. Other forms of actuators or other methods of use of actuators may be utilized in examples.

The prosthetic valve 169 may be configured similarly as other forms of prosthetic valves disclosed herein (including prosthetic valve 10) unless stated otherwise. The prosthetic valve 169, for example, may include a support structure 186, which may include a support frame 188. The support frame 188 may include an outer support stent and/or an inner support stent in examples. The support structure 186 may support a valve portion that may be configured similarly as other forms of valve portions disclosed herein, including use of a plurality of prosthetic valve leaflets. The support structure 186 may include a sealing skirt 245 in examples. One or more anchors 247 may be utilized that may be configured similarly as the anchors 17 or may have other forms in examples.

The actuators 163 and inflatable bodies 167 are preferably positioned upon the support structure 186 in a location to apply the vibrations to the calcification 84, 86 from the outer surface 179 of the support structure 186. The outer surface 179 of the support structure 186 may be for contact with the calcification of the native valve. The actuators 163 and inflatable bodies 167, for example, may be positioned between the support frame 188 and the sealing skirt 245 in examples. The sealing skirt 245 may overlay the inflatable bodies 167. The inflatable bodies 167 may transmit the vibrations through the scaling skirt 245 and to the calcification 86, 84. In examples, the actuators 163 and inflatable bodies 167 may be positioned radially outward of the sealing skirt 245 for application of the vibrations to the calcification 84, 86. For example, the one or more inflatable bodies 167 may comprise a tube or ring extending around the sealing skirt 245. In examples, the one or more inflatable bodies 167 may comprise a tube or ring extending around the support frame 188 and interior of the sealing skirt 245. Other configurations of the one or more inflatable bodies 167 or locations of the one or more inflatable bodies 167 may be utilized in examples.

The one or more inflatable bodies 167 may be provided in an inflated state upon deployment or may be filled via one or more fluid conduits from a delivery system or delivery catheter upon implantation. The one or more inflatable bodies 167 may remain inflated following implantation or may be drained of fluid after their use for removal of the calcification.

The actuators 163 may include one or more electric terminals 181 that may be configured for providing electrical energy to the actuators 163. The electric terminals 181 may electrically couple to the actuators 163 via one or more electrical conduits 183 that may extend along the support structure 186 between the actuators 163 and the electric terminals 181.

The delivery system or delivery catheter may include an actuation mechanism 185 for transmitting energy to the support structure 186 to cause the support structure 186 to produce the vibrations for reducing calcification of the calcified native valve. The actuation mechanism 185 may include one or more electric terminals 187 for electrical conduction with the one or more electric terminals 181 of the actuators 163. The electric terminals 187 may extend to a controller such as a controller 128 shown in FIG. 6C (which may be powered by a power source 131) to control the actuators 163 in a similar manner as the controller 128. The controller may control energy that is transmitted to the support structure 186 to cause the support structure to vibrate. The electric terminals 187 may couple to electrical conduits 189, 191 that may extend to the controller in a similar manner as the electrical conduits 120, 122. The electrical conduits 189, 191 may extend along an elongate shaft of a delivery system or delivery catheter in examples. The electric terminals 187 may comprise releasable terminals that may release from the electric terminals 181 following an implantation procedure. For example, the electric terminals 181 may be magnetically coupled or coupled with a releasable clip, clamp, or other form of coupler to the electric terminals 181. As such, the electric terminals 187 and electrical conduits 189, 191 may be removed following implantation.

The actuators 163 may be operated to break up calcification in a similar manner as discussed regarding the actuators 123. Electrical energy may be provided to the actuators 163 in a controlled manner via a controller (which may be the controller 128). A desired amount of calcification may be reduced in the process. In examples, a sensor 133 may be provided that may be used to image the reduction of the calcification. The sensor 133 may operate similarly as disclosed herein. For example, the sensor 133 may image the diameter of the prosthetic valve 169. An output from the sensor 133 may be provided as feedback to the controller for the controller to automatically produce the vibrations from the one or more inflatable bodies 167 and/or cease producing the vibrations upon a sufficient amount of the calcification being removed. Other configurations may be utilized in examples.

A resulting configuration of the prosthetic valve 169 is shown in FIG. 7G.

In examples, the use of the actuators 163 may be combined with a configuration as represented in FIG. 7D, in which current is applied to the support frame to control an expansion of the support frame. The actuators 163 may be operated to reduce the calcification of the native valve and the current may be applied to the support frame to increase the diameter of the support frame. In examples, the actuators 163 may be utilized solely.

The form of the actuators utilized may be varied in examples.

FIGS. 7H and 7I, for example, illustrate a variation in which one or more actuators 195 may comprise piezoelectric actuators. The actuators 195 may be positioned in similar locations as disclosed with the actuators 163 and inflatable bodies 167. The actuators 195, for example, may be positioned to apply vibrations to calcification from an outer surface 197 of the support structure 199 of the prosthetic valve 201. The prosthetic valve 201 may otherwise be configured similarly as the prosthetic valve 169.

The piezoelectric actuators may include a piezoelectric material 251 and a pressing surface 261 for applying the vibrations to the calcification produced by the piezoelectric material 251. A voltage applied to the piezoelectric material 251 and supplied from the electric terminals 263 via the electrical conduits 265 may be utilized. A controller (similar to controller 128) and power source 131 may provide the electrical energy to the piezoelectric material 251 to produce the vibrations at the desired frequency (which may be an ultrasonic frequency). The actuators 195 may be utilized to reduce the calcification in a similar manner as disclosed regarding other forms of actuators described herein.

In examples, a sensor 133 may be utilized for feedback to the controller in a similar manner as disclosed herein. In examples, the piezoelectric actuator may be utilized as a sensor for determining an amount of force applied to the calcification. Such sensor signals may be utilized as feedback to the controller to determine a force applied to the calcification. The controller may control the operation of the piezoelectric actuator based on the feedback received from the piezoelectric actuator (e.g., vary frequency of the vibrations or cease operation of the piezoelectric actuator based on the feedback). FIG. 7I illustrates a resulting configuration of the prosthetic valve 201 in examples. The electric terminals 211 from the actuation mechanism of the delivery system or delivery catheter may be removed upon deployment.

The use of the actuators 195 may be combined with a configuration as represented in FIG. 7D, in which current is applied to the support frame to control an expansion of the support frame. The actuators 195 may be operated to reduce the calcification of the native valve and the current may be applied to the support frame to increase the diameter of the support frame. In examples, the actuators 195 may be utilized solely.

The vibrations produced in FIGS. 7F-71 may comprise acoustic pressure waves in examples. The acoustic pressure waves may comprise ultrasonic waves in examples. The ultrasonic waves may comprise shock waves in examples. Various other forms or frequencies of waves may be utilized to produce a desired result in examples (e.g., low frequency waves at less than an ultrasonic frequency may be utilized in examples, and higher frequency waves may be utilized).

Other forms of actuation mechanisms for transmitting energy to a support structure to cause the support structure to produce vibrations for reducing calcification of the calcified native valve may be provided in examples.

FIGS. 7J-7L, for example, illustrate variations in which an actuation mechanism is adapted to apply vibrations to a support frame 215 to cause the support frame 215 to vibrate, thereby reducing calcification of the calcified native valve. The support frame 215 may transmit the vibrations for reducing calcification of the calcified native valve. The actuation mechanism may include one or more forms of actuators disclosed herein, or other forms of actuators.

For example, referring to FIG. 7J, the actuation mechanism 221 may include an actuator in the form of electrodes 223 that may be configured to excite or vaporize a fluid filling an inflatable body 225 to produce the vibrations of the inflatable body 225 in a similar manner as the actuators 123. For example, the electrodes 223 may produce an electrical arc or spark upon a voltage being applied to the electrodes 223. The electrical arc or spark may vaporize a part of the fluid filling the inflatable body 225 (e.g., a portion of the fluid adjacent to the electrodes 223), with the gas pressure of vaporization producing the vibrations of the inflatable body 225. The vibrations may propagate through the fluid to the outer surface of the inflatable body 225 and apply the vibrations to support frame 215. Other forms of actuators or other methods of use of actuators may be utilized in examples.

The actuation mechanism 221 may include a support 277 such as a control arm or other form of support for controlling the position of the inflatable body 225.

The inflatable body 225 may be applied to the support frame 215 to apply the vibrations to the support frame 215. The inflatable body 225, for example, may be positioned against a proximal end portion or inlet end portion 229 of the prosthetic valve 231. Other application locations (e.g., a distal end portion or outlet end portion) may be utilized as desired. The inflatable body 225 may contact the support frame 215 or may be applied to a sealing skirt, with the vibrations transmitting to the support frame 215 through the sealing skirt.

The vibrations applied to the support frame 215 may propagate through the support frame 215 and apply the vibrations or shock waves to the calcification of the native valve. The vibrations or shock waves may reduce the calcification of the native valve.

The prosthetic valve 231 may be configured similarly as other forms of prosthetic valves disclosed herein, including the prosthetic valve 10 or other forms of prosthetic valves. The vibrations may propagate along an outer support stent for direct application to the calcification or another portion of the support frame as desired.

In examples, the actuation mechanism 221 may comprise a portion of the delivery system or delivery catheter utilized to deploy the prosthetic valve 231. The support 277, for example, may extend from the delivery catheter to apply the inflatable body 225 to the support frame 215. A separate device may apply the inflatable body 225 to the support frame 215 in examples. The operation of the actuator may be controlled via a controller utilizing methods disclosed herein. Feedback from a sensor 133 to the controller may further be utilized according to methods disclosed herein.

The use of the actuation mechanism 221 may be combined with a configuration as represented in FIG. 7D, in which current is applied to the support frame to control an expansion of the support frame. The actuator may be operated to reduce the calcification of the native valve and the current may be applied to the support frame to increase the diameter of the support frame. In examples, the actuation mechanism 221 may be utilized solely. A resulting configuration of the prosthetic valve 231 is shown in FIG. 7K.

The configuration of the actuator utilized in FIG. 7J may be varied as desired. FIG. 7L, for example, illustrates use of an actuation mechanism 279 in the form of a piezoelectric actuator 281 for applying the vibrations to the support frame 215. The actuation mechanism 279 may otherwise operate in a similar manner as disclosed regarding the actuation mechanism 221.

Variations in the systems and methods may be utilized in examples. The vibrations produced in FIGS. 7J-7L may comprise acoustic pressure waves in examples. The acoustic pressure waves may comprise ultrasonic waves in examples. The ultrasonic waves may comprise shock waves in examples. Various other forms or frequencies of waves may be utilized to produce a desired result in examples (e.g., low frequency waves at less than an ultrasonic frequency may be utilized in examples, and higher frequency waves may be utilized).

The systems, methods, and apparatuses may be utilized for a mitral valve or a triscupid valve, or other forms of valves as desired. The features of the examples FIGS. 7A-7L may be utilized solely or in combination with the features of any other example herein.

Variations in the systems and methods disclosed herein may be provided.

FIG. 8 illustrates a system in which a cutter 151, such as a sintering device or laser may be utilized to reduce calcification of a native heart valve. The cutter 151 may be positioned on a catheter or may be otherwise positioned and may be movable to be placed in a desired position relative to calcification. The cutter 151 may be utilized to reduce the calcification by smoothing the calcification or fracturing the calcification as desired. For example, FIG. 8 illustrates a cutter 151 in the form of a laser that is fracturing calcification 157 for removal from the implantation site.

The cutter 151 may be utilized to provide a desired shape of the calcification or may be utilized to partially or entirely remove the calcification. For example, the cutter 151 may be utilized to cut protruding portions 159 of calcification 157 to reduce the possibility of such protruding portions 159 interfering with deployment of a prosthetic valve. Thick or jagged calcification may be cut or smoothed. In examples, sections of calcification or the entirety of calcification of a native valve may be cut or smoothed.

In examples, an embolic capture device 168a may be utilized that may capture calcification that releases from the native valve. The embolic capture device 168a, for example, may comprise a filter (e.g., a body having pores) that may allow blood to pass through and capture the calcification. The filter may be shaped as a receptacle that may retain the released calcification. The calcification retained by the embolic capture device 168a may be removed following the reduction of calcification of the native heart valve. In examples, the embolic capture device 168a may comprise a first embolic capture device 168a, and a second embolic capture device 168b may be utilized. The embolic capture device 168b may be positioned upon an upstream or atrial side of the native valve and the embolic capture device 168a may be positioned on a downstream or ventricular side of the native valve. The embolic capture device 168b may be coupled to the cutter 151 and may extend radially outward from the cutter 151 or another configuration may be utilized in examples. In examples, only one of the devices 168a, 168b may be utilized.

The calcification may be removed from a mitral valve or a tricuspid valve or another form of valve as desired. In examples, a prosthetic heart valve may be deployed to the native heart valve as desired.

The features of the examples of FIG. 8 may be utilized solely or in combination with the features of any other example herein.

In examples, anchors that may be utilized to anchor a prosthetic valve to an implantation site may be customized according to a configuration of an implantation site at a native heart valve having calcification. For example, the configuration of the implantation site may be determined. The determination may be made in a variety of manners. In examples, the configuration may be determined based on imaging of the implantation site. In examples, the configuration may be determined based on demographic or statistical information of a patient (e.g., age, weight, medical history, among others). A configuration of a distal anchor of a prosthetic heart valve may be selected based on the determined configuration of the implantation site.

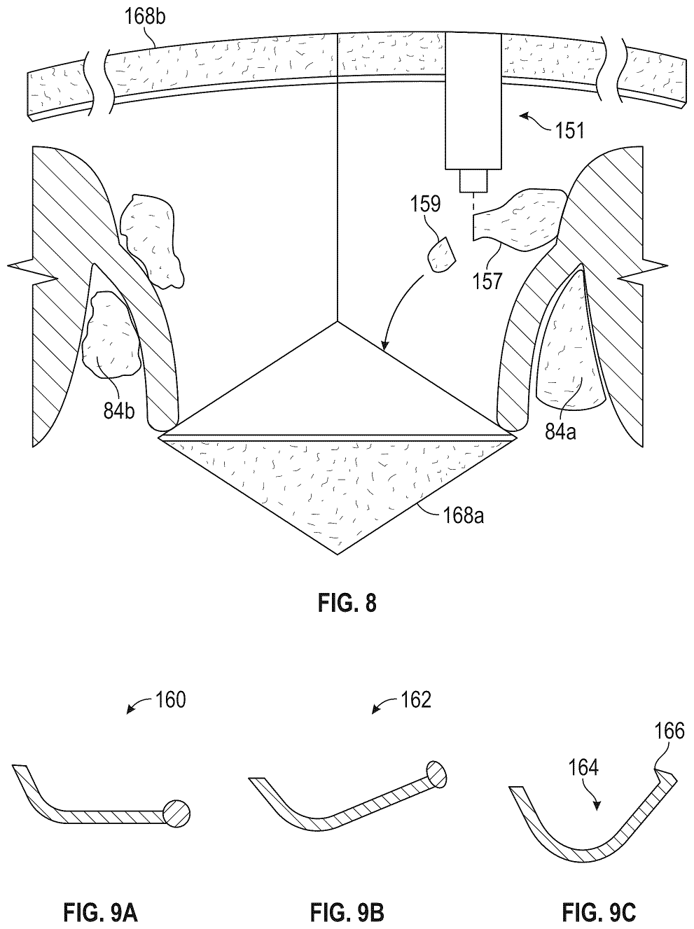

For example, FIGS. 9A-9C illustrate variations of a distal anchor that may be selected. FIG. 9A illustrates a distal anchor 160 that may extend horizontally to be positioned distal of calcification and to not hook over native valve leaflets. The anchor 160 may extend over a distal tip of the leaflet. Such an anchor 160 may be selected if it is determined that an implantation site has calcification 84a radially outward of a leaflet as shown in FIG. 8 for example. FIG. 9B illustrates an example of an anchor 162 that may be angled proximally to account for partial calcification 84b positioned radially outward of a leaflet as shown in FIG. 8 for example. FIG. 9C illustrates an example of an anchor 164 including a barb 166 for engaging calcification as desired.

The configuration of the distal anchor may be selected based on the determined configuration of the implantation site. The configuration of the distal anchor may be selected and then the selected anchor may be attached to a support structure, or an entire prosthetic valve may be selected to provide the desired configuration of the selected anchor. The prosthetic valve may be deployed with the anchor having the selected configuration. The configuration of the distal anchor may be selected based on the shape of calcification at the native heart valve. The configuration of the distal anchor may be selected to anchor to the calcification at the native heart valve.

In examples, the configuration of the distal anchor may be selected from a set including a plurality of different configurations of distal anchors.

In examples, a distal anchor may be adjusted based on the selected configuration. For example, an anchor 162 as shown in FIG. 9B may be adjusted to extend horizontally as shown in FIG. 9A to account for calcification as desired. The anchor 162 may be bent to extend horizontally from the configuration shown in FIG. 9B to the configuration shown in FIG. 9A. An adjustment of an angle of a distal anchor may be provided relative to a frame of the support structure.

The anchors may comprise distal anchors that may be coupled to a support structure 15 as shown in FIGS. 1A-IC and may be utilized in lieu of or in combination with the anchors 17 shown in FIGS. 1A-1C. In examples, the anchors may couple to other forms of support structures. The prosthetic valve may be configured to be deployed to a mitral valve or a tricuspid valve in examples.

Various other configurations of distal anchors may be utilized in examples. The features of the examples of FIG. 9A-9C may be utilized solely or in combination with the features of any other example herein.

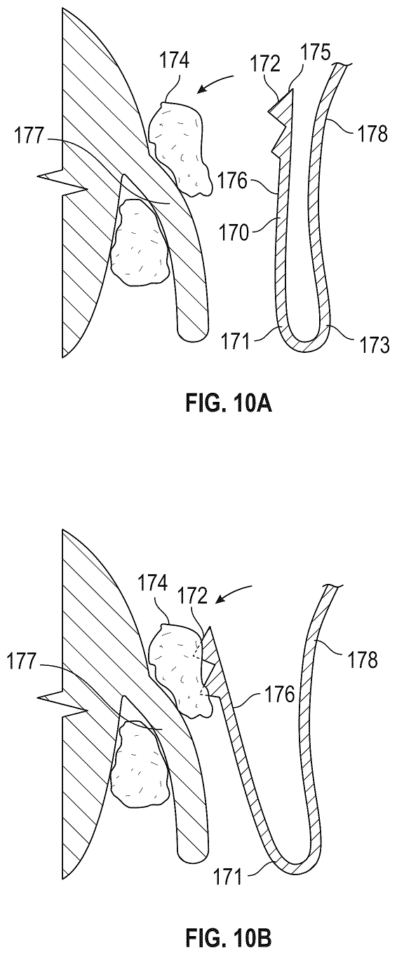

FIGS. 10A-10B illustrate an example of an anchor 170 that may include a barb 172 that may be configured to engage calcification 174 of the native valve to anchor to the calcification 174. The barb 172 may be positioned on an arm 176 that may be configured to extend radially outward from a support structure 178 to engage the calcification.

The arm 176 may have a distal end portion 171 coupled to a distal end portion 173 of the support structure 178. The arm 176 may extend proximally from the distal end portion 171 to a proximal end portion 175 of the arm 176. The arm 176 may extend proximally radially outward of a support structure 178 (which may be configured similarly as the support structure 15 shown in FIGS. 1A-1C). The arm 176 may be configured to be positioned interior of the inward facing surfaces of the native valve leaflets 177 and rotate outward from the support structure 178 to engage the calcification 174.

For example, referring to FIG. 10B, the arm 176 may pivot about the distal end portion 171 to move radially outward from the support structure 178. The arm 176 may move radially outward for the barb 172 to engage the calcification 174. The arm 176, including the proximal end portion 175, may be positioned interior of the inward facing surface of the native valve leaflet 177 upon anchoring to the calcification.

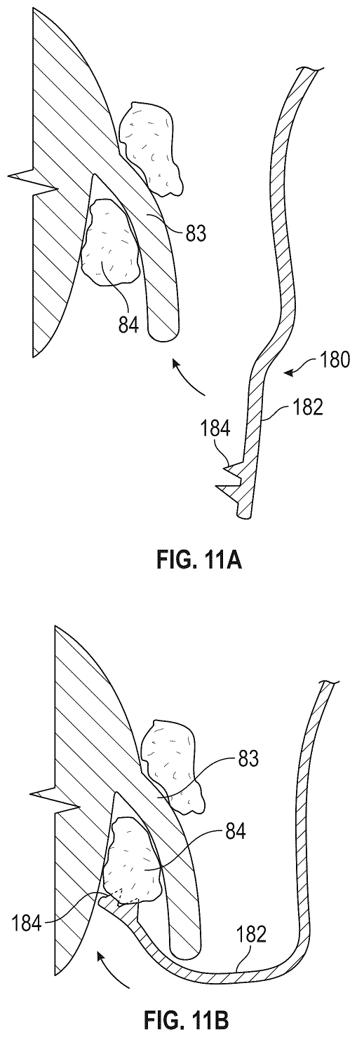

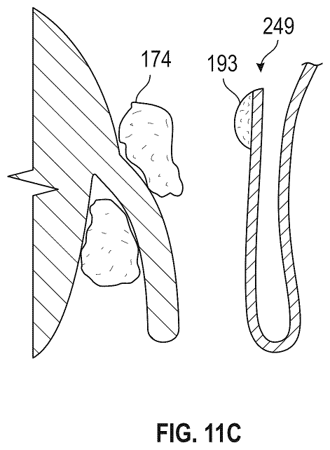

In examples, the configuration of the anchor may be varied. For example, FIGS. 11A-11B illustrate an example of an anchor 180 having an arm 182 that may be configured to rotate proximally to engage calcification 84 that may be positioned radially outward of an inward facing surface of a heart valve leaflet 83 upon anchoring to the calcification. FIG. 11B, for example, illustrates the rotation of the arm 182 to allow the barb 184 to engage the calcification 84. The arm 182 may be positioned radially outward of the native valve leaflet 83.

Referring to FIG. 11C, in examples, an anchor 249 may include a pad 193. The pad 193 may comprise a conformable body configured to conform to a shape of calcification 174. The pad 193, for example, may comprise a cloth body or an inflatable body. Other forms of pads may be utilized in examples. A pad may be utilized with an anchor 170 as shown in FIGS. 10A-10B or an anchor 180 as shown in FIGS. 11A-11B.

The anchors may comprise distal anchors that may be coupled to a support structure 15 as shown in FIGS. 1A-1C and may be utilized in lieu of or in combination with the anchors 17 shown in FIGS. 1A-1C. The anchors, and the proximal end portions of the anchors, may be configured to be biased outward from the support structure. The support structure may support one or more prosthetic valve leaflets positioned within a flow channel or passageway. At least one anchor may couple to the support structure and may include one or more of a barb or a pad configured to engage calcification of the native valve to anchor to the calcification. In examples, the anchors may couple to other forms of support structures.

In examples, other forms of anchoring may be utilized. The features of the examples of FIGS. 10A-11C may be utilized solely or in combination with the features of any other example herein.

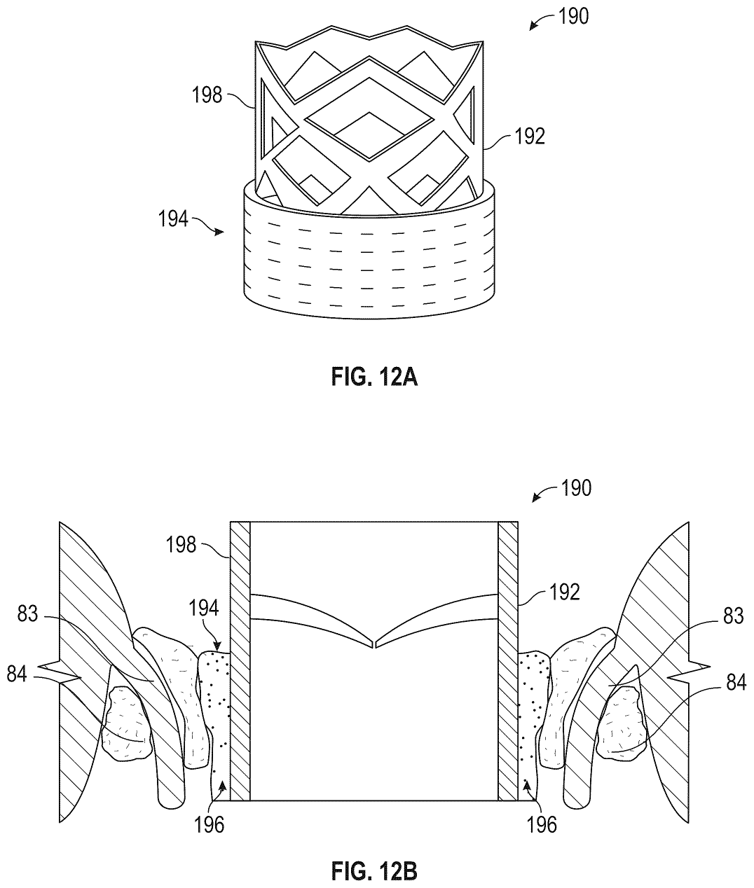

FIGS. 12A-12B illustrate an example of a prosthetic valve 190 including a support structure 192 having an outer surface 194 comprising a conformable anchoring surface configured to conform to a shape of calcification of the native valve to anchor to the native valve.

The support structure 192, for example, may include a conformable outer cloth, or may comprise a fluid. The support structure 192 may include a chamber 196, for example, that may be configured to retain the fluid. The chamber 196 may have a flexible wall comprising the outer surface 194 of the support structure 192. The outer surface 194 accordingly may conform to a shape of a native valve that may include calcification, as shown in FIG. 12B for example. The conformable anchoring surface may be configured to deflect radially inward to conform to a shape of calcification.

The fluid may comprise a liquid (such as saline) or may comprise a hydrogel or other form of gel in examples. Other forms of fluid may be utilized in examples. Other forms of fill materials disclosed herein may be utilized.

The chamber 196 may be positioned upon a frame 198 of the support structure 192. The frame 198 accordingly may be positioned radially inward of the conformable anchoring surface of the chamber 196. The chamber 196 may cover the entirety of the outer surface of the frame 198 or only a portion (as shown in FIG. 12A for example).

In examples, the outer surface 194 may comprise the anchoring surface for the prosthetic valve 190 and the prosthetic valve 190 may lack additional anchors (e.g., distal anchors or atrial anchors). As such, a reduced possibility of interference of such additional anchors with calcification positioned radially outward of native valve leaflets 83 may result.

The support structure 192 may be configured to support one or more prosthetic valve leaflets that are configured to be positioned in a flow channel or passageway.

The features of FIGS. 12A and 12B may be utilized solely or in combination with the features of any other example herein.

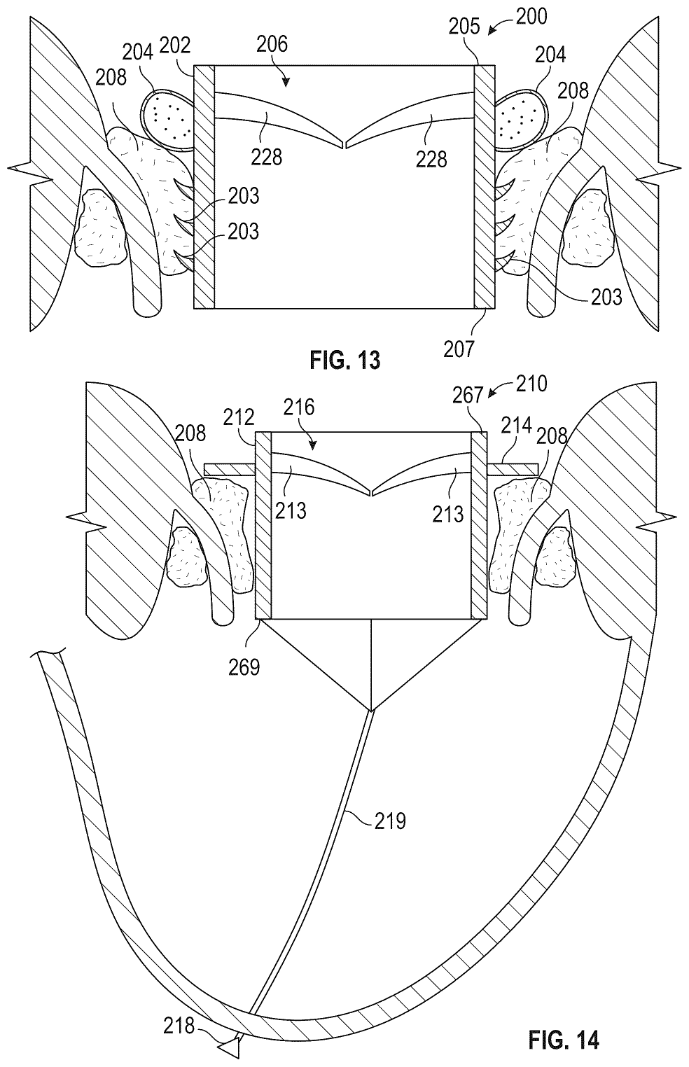

FIG. 13 illustrates an example of a prosthetic valve 200 including a support structure 202 having an atrial anchor 204 comprising a flange configured to extend radially outward from the flow channel or passageway 206 of the prosthetic valve 200. The atrial anchor 204, in examples, may comprise an inflatable body forming a ring about the passageway 206. The inflatable body may be configured to conform to a shape of calcification 208 that may be present at the native heart valve.

The inflatable body may be filled with fluid in examples, and may be filled in vivo or may be filled prior to insertion into a patient's body in examples. The inflatable body may be configured to form a fluid seal with the calcification 208 in examples.

In examples, the fill material may comprise a hardenable material. The fill material may be configured to harden over time to enhance the sealing of the inflatable body. The hardenable material that may be introduced into an inflatable body at a first, relatively low viscosity and converted to a second, relatively high viscosity. Viscosity enhancement may be accomplished through a variety of UV initiated or catalyst-initiated polymerization reactions, or other chemical system. The end point of the viscosity enhancing process may result in a hardness anywhere from a gel to a rigid structure, depending on the desired performance.

A hardenable material may comprise an epoxy. The epoxy may be hardened by mixing materials that harden when combined. The hardening catalyst may be delivered during implantation or later. The hardenable material may be biocompatible and able to conform to the shape of the local native valve. In embodiments, the hardenable material may be bioresorbable.

In examples, the fill material may be radiopaque for visualization during implantation. A radiopaque material may be added during filling, as part of a hardening process for example.

In examples, the fill material may comprise a gel or a foam, which may be biocompatible, and may be configured to harden over time. A gel or foam may be inserted into the inflatable body or may be provided in capsules that dissolve upon implantation to allow for expansion.

In embodiments, a gel may be utilized that may be made via polymer precipitation from biocompatible solvents. Various siloxanes may be utilized as inflation gels as well. Other gel systems that may be utilized may include phase change systems that gel upon heating or cooling from their initial liquid or thixotropic state. Gels may also comprise thixotropic material that undergo sufficient shear-thinning so that they may be readily injected through a fluid conduit yet are also gel-like at zero or low shear rates.

In embodiments, a fill material may contain a foaming agent. The foaming agent may generate pressure within the inflatable body.

Any of the fill materials disclosed herein may be biocompatible in embodiments and may be bioresorbable if desired. A bioresorbable sealing body may improve sealing through tissue adhesion with the native valve.

The atrial anchor 204 may be configured to resist a distal or ventricular force applied to the prosthetic valve 200.

The support structure 202 may support a valve portion as disclosed herein. For example, the support structure 202 may support one or more prosthetic valve leaflets 228 that may be positioned within the passageway 206. The support structure 202 may have an inlet end portion 205 and an outlet end portion 207 as disclosed herein.

In examples, the support structure 202 may further include a plurality of barbs 203 that may extend radially outward from the passageway 206. The plurality of barbs 203 may protrude from the outer surface of the support structure 202 and may anchor the prosthetic valve 200 in position. The barbs 203 may be configured to resist a proximal or atrial force applied to the support structure 202. For example, the barbs 203 may be angled proximally in examples. In examples, the atrial anchor 204 may be excluded and the barbs 203 may be utilized solely.

The features of FIG. 13 may be utilized solely or in combination with the features of any other example herein. Various other forms of anchors may be utilized in examples.

FIG. 14 illustrates an example of a prosthetic valve 210 including a support structure 212 having an atrial anchor 214 comprising a flange configured to extend radially outward from the flow channel or passageway 216 of the prosthetic valve 210. The atrial anchor 214, in examples, may comprise a shelf forming a ring about the flow channel or passageway 216. The shelf, in examples, may include a sealing surface that may be configured to seal with calcification 208 of a native valve, and conform to a shape of the calcification 208 that may be present at the native heart valve. The support structure 212 may support a valve portion as disclosed herein. For example, the support structure 212 may support one or more prosthetic valve leaflets 213 that may be positioned within the passageway 216. The support structure 212 may have an inlet end portion 267 and an outlet end portion 269 as disclosed herein.

The atrial anchor 214 may be configured to resist a distal or ventricular force applied to the prosthetic valve 210.

In examples, the support structure 212 may be coupled to an anchor 218 that may be configured to anchor to a ventricular wall. The anchor 218 may be configured to anchor to an apex of a ventricle or to another portion of a ventricle as desired. The anchor 218 may be positioned exterior of a ventricular wall or may be engaged with an inner surface of the ventricular wall as desired. In examples, the anchor 218 may be deployed transcatheter and a ventricular approach may be utilized. For example, a delivery apparatus may approach from the mitral valve to the implantation site on the ventricle. In examples, other approaches may be utilized (e.g., a transapical approach).

The anchor 218 may couple to the support structure 212 with a tether 219. The tether 219 may comprise a compliant body that may allow for tension to be applied to the tether without causing the heart to deform. The tether 219 may comprise a cord, a wire, or a braid, or may have another form as desired. The tether 219 may comprise a shape memory material such as Nitinol or may have another form as desired. The anchor 218 and tether 219 may resist a force applied in a proximal or atrial direction applied to the support structure 212.

The features of FIG. 14 may be utilized solely or in combination with the features of any other example herein.

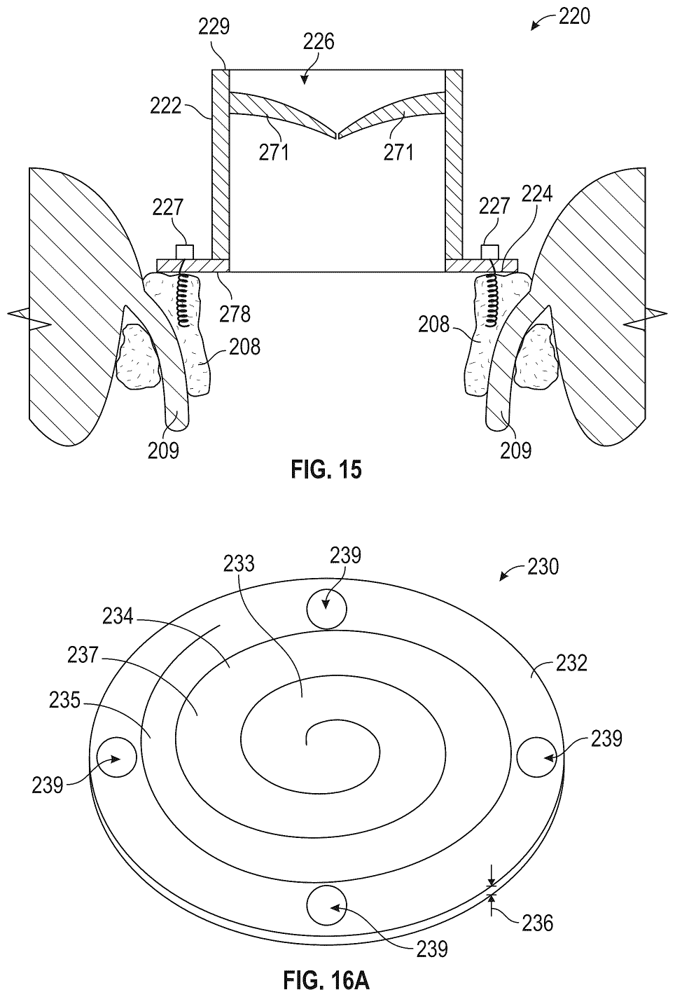

FIG. 15 illustrates an example of a prosthetic valve 220 including a support structure 222 having an atrial anchor 224 comprising a flange configured to extend radially outward from the flow channel or passageway 226 of the prosthetic valve 220. The atrial anchor 224, in examples, may comprise a shelf forming a ring about the passageway 226. The shelf, in examples, may include a sealing surface that may be configured to seal with calcification 208 of a native valve, and conform to a shape of the calcification 208 that may be present at the native heart valve.

The atrial anchor 224 may be configured to resist a distal or ventricular force applied to the prosthetic valve 220. In examples, one or more penetrating bodies 227 may be utilized that may anchor the prosthetic valve 220 to the heart valve. For example, the one or more penetrating bodies 227 may comprise screws, barbs, or clips, that may anchor to the heart valve, which may include anchoring to the calcification 208. The one or more penetrating bodies 227 may be configured to pass through the atrial anchor 224 comprising a flange to anchor the support structure to the native valve.

The support structure 222 may extend proximally or in an atrial direction from the atrial anchor 224. For example, the support structure 222 may be positioned supra-annularly to reduce the possibility of interference with the native valve leaflets 209. One or more prosthetic valve leaflets 271 may be positioned proximal of the atrial anchor 224.

The support structure 222 may support a valve portion as disclosed herein. For example, the support structure 222 may support the one or more prosthetic valve leaflets 271 that may be positioned within the passageway 226. The support structure 222 may have an inlet end portion 229 and an outlet end portion 278 as disclosed herein.

The features of FIG. 15 may be utilized solely or in combination with the features of any other example herein.

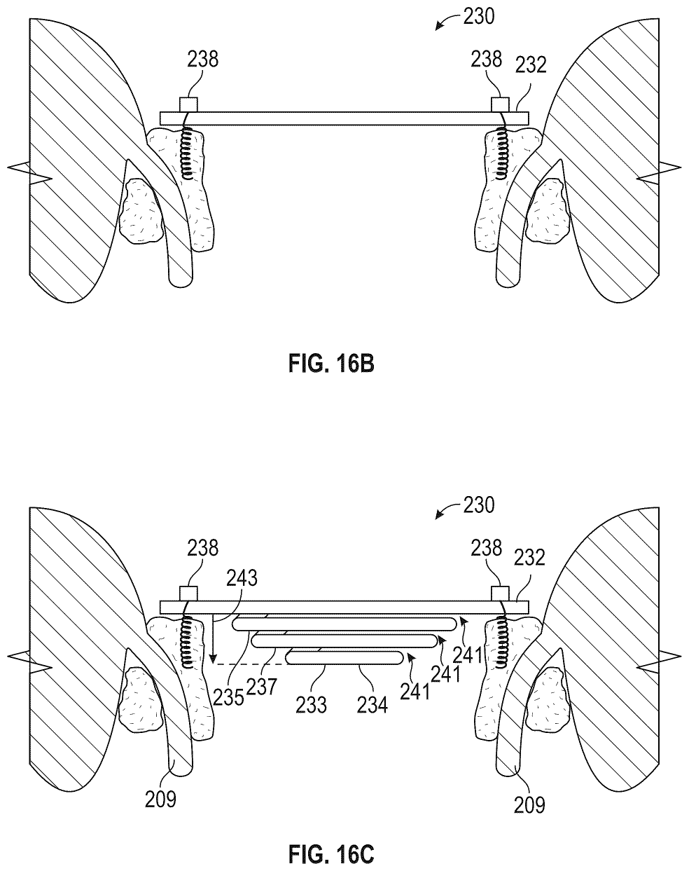

FIGS. 16A-16C illustrate an example of a prosthetic valve 230 including a support structure 232 and a spiral body 234 coupled to the support structure 232 and configured to move between an opened state and a closed state to control fluid flow or blood flow through the support structure 232. The support structure 232 may include a passageway shown closed in FIG. 16A, yet shown open in FIG. 16C. FIG. 16A illustrates the spiral body 234 in a closed state.

The support structure 232 may comprise a ring that may extend around the spiral body 234 and may be configured to be positioned on an atrial side of the heart valve. The ring may comprise a flattened ring with a thin profile, and the thickness 236 of the ring may be the same as a thickness of the spiral body 234 in examples. In examples, the ring may have a different thickness than the spiral body 234. The ring may be configured to be anchored to the valve annulus with one or more penetrating bodies 238 (marked in FIG. 16B). The penetrating bodies 238 may be configured to pass through anchoring portions 239 of the support structure 232 to anchor to the valve annulus of the native valve.

The spiral body 234 may comprise an arm forming a spiral and having a radially inward portion 233 and a radially outward portion 235 positioned radially outward of the portion 233. An intermediate portion 237 may be positioned between the radially inward portion 233 and the radially outward portion 235. One or more wraps of the arm may form the spiral configuration. The radially outward portion 235 may have a greater diameter than the intermediate portion 237, which may have a greater diameter than the radially inward portion 233. The radially outward portion 235 may be coupled to the support structure 232, and the radially inward portion 233 and the intermediate portion 237 may move distally relative to the support structure 232.

For example, referring to FIG. 16B, the prosthetic valve 230 may be in a closed state, with the spiral body 234 in a configuration as shown in FIG. 16A. The edges of the spiral body 234 may be in contact with each other to form a seal of the valve. The passageway of the support structure 232 may be closed. The radially inward portion 233 may be coplanar with the radially outward portion 235 in the closed state.

Upon diastolic pressure applied to the spiral body 234, the radially inward portion 233 may extend distally or in a ventricular direction, along with the intermediate portion 237 and the radially outward portion 235. The spiral body 234 may move distally to move from the closed state to the opened state. The radially inward portion 233 may be distal of the radially outward portion 235 in the opened state. Gaps 241 may exist between the portions 233, 237, 235 that may allow fluid flow therethrough. The gaps 241 may be formed when the spiral body 234 moves to the opened state. Upon systolic pressure applied to the prosthetic valve 230, the spiral body 234 may move to the closed state as shown in FIG. 16B. The gaps 241 may be closed when the spiral body 234 is in the closed state. The force of the fluid (e.g., blood) may move the spiral body 234 between the opened state and the closed state. The spiral body 234 may cyclically move between the opened state and the closed state.

A distance 243 that the spiral body 234 opens to may be varied in examples. For example, referring to FIG. 16C, the distance 243 may be less than a length of a native valve leaflet 209 that is in an opened state. The distance 243 may be varied in embodiments. For example, FIG. 17A illustrates an example of a prosthetic valve 240 including a spiral body 242 that may extend to a distance 244 that may be at or proximal a length of the native valve leaflet 209 that is in an opened state. The spiral body 242 may retract proximally or in an atrial direction to close the prosthetic valve 240. In examples, as shown in FIG. 17B, the spiral body 242 may retract and may avoid impeding or contacting the leaflets 209 as they move to a closed state.

In the example of FIGS. 17A-17B, the support structure 246 may comprise a ring that may anchor to an atrial wall. One or more penetrating bodies 248 may anchor to the atrial wall. The penetrating bodies 248 may comprise screws, barbs, or clips. In examples, one or more sutures may be utilized to anchor the support structure 246 to the atrial wall.

The prosthetic valves disclosed herein may be deployed to a mitral valve or a tricuspid valve or another form of valve as desired. Other forms of prosthetic valves and anchors may be utilized in examples. The features of FIGS. 16-17B may be utilized solely or in combination with the features of any other example herein.

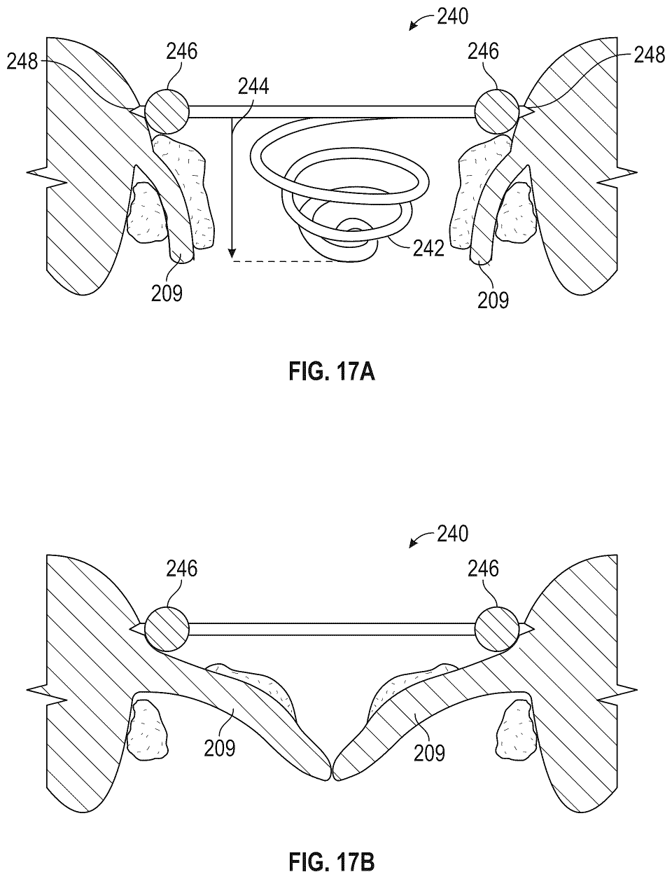

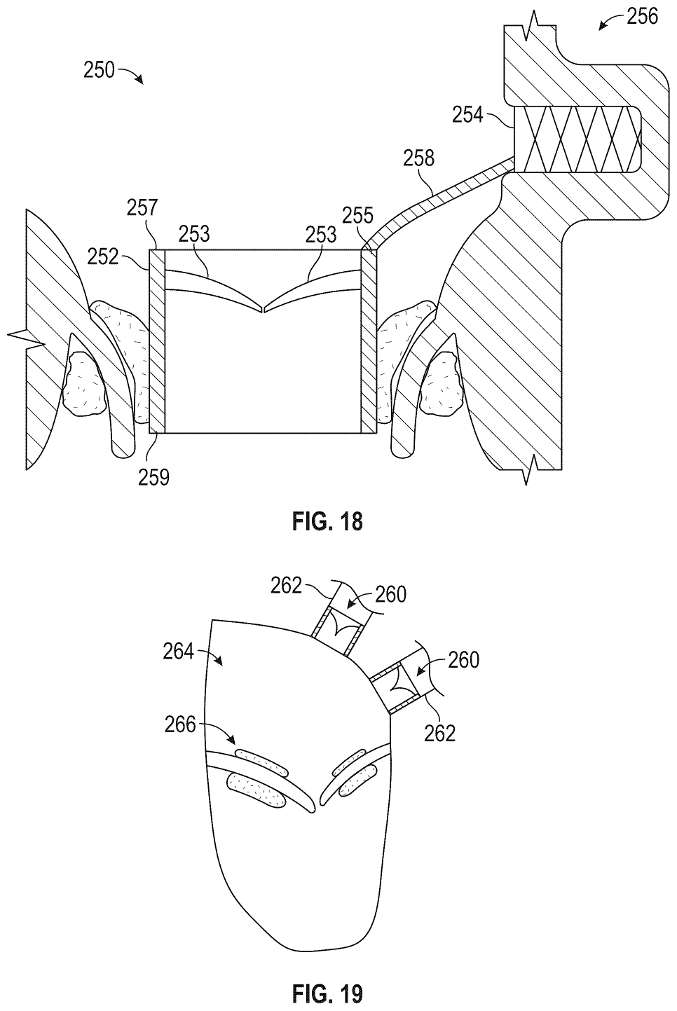

FIG. 18 illustrates an example of a system 250 for a heart. The system 250 may include a prosthetic heart valve 252 configured to be implanted in a mitral valve of the heart. An anchor 254 may be coupled to the prosthetic heart valve 252 and configured to be implanted in a left atrial appendage 256 of the heart. The anchor 254 may be configured to retain the prosthetic valve leaflet in position in the mitral valve of the heart. The anchor 254 is coupled to the prosthetic heart valve 252 for anchoring the prosthetic heart valve 252 within the native mitral valve.

In examples, the anchor 254 may comprise a stent that may be configured to be deployed to the left atrial appendage 256. The stent may be inserted into the left atrial appendage 256 and may anchor to the left atrial appendage 256. In examples, the anchor 254 may have other forms as desired.

A tether 258 may couple the anchor 254 to the prosthetic heart valve 252. The tether 258 may extend within a left atrium of the heart. The tether 258 may comprise a rigid body that may retain the prosthetic heart valve 252 in position within the mitral valve.

The prosthetic heart valve 252 accordingly may lack anchors directly to the mitral valve or calcification of the mitral valve as the anchor 254 may provide the anchoring for the prosthetic heart valve 252. The prosthetic heart valve 252 may include one or more prosthetic valve leaflets 253 coupled to a support structure 255. The support structure 255 may support a valve portion as disclosed herein. For example, the support structure 255 may support one or more prosthetic valve leaflets 253 that may be positioned within a passageway of the support structure 255. The support structure 255 may have an inlet end portion 257 and an outlet end portion 259 as disclosed herein. The support structure 255 may be configured similarly as other forms of support structures disclosed herein.

The features of FIG. 18 may be utilized solely or in combination with the features of any other example herein. Other forms of prosthetic valves and anchors may be utilized in examples.

FIG. 19 illustrates an example in which one or more prosthetic valves 260 are implanted within pulmonary veins 262. The implantation may impede fluid flow to a lung. The implantation may prevent regurgitation from entering the lungs. A reduced fluid buildup within the lungs may occur. The prosthetic valves 260, for example, may be configured with a frame coupled to one or more prosthetic valve leaflets to impede fluid flow to the lungs and may allow fluid flow from the lungs to the left atrium 264. The prosthetic valves 260 may be configured to open in a flow direction towards a left atrium of the heart. Such a configuration may be utilized in circumstances in which the mitral valve 266 has calcification and a prosthetic valve may not be implanted therein. One or multiple prosthetic valves may be implanted to one or more pulmonary veins 262. In examples, a configuration as shown in FIG. 19 may be utilized in combination with a prosthetic mitral heart valve implanted to the mitral valve.

The features of FIG. 19 may be utilized solely or in combination with the features of any other example herein. Other forms of prosthetic valves and anchors may be utilized in examples.

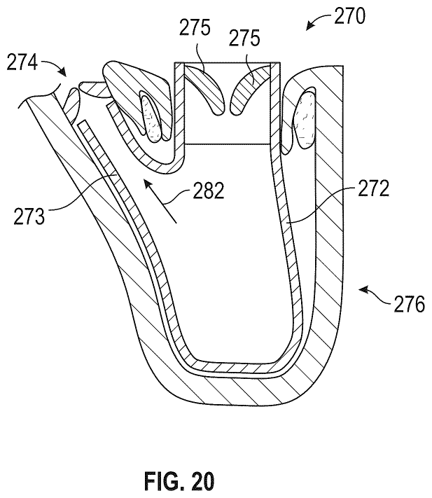

FIG. 20 illustrates an example including a prosthetic heart valve 270 and an anchor 272 coupled to the prosthetic heart valve 270 and comprising a ventricular chamber configured to extend within a ventricle of the heart. The prosthetic heart valve 270 may be configured to be implanted in a valve of the heart, which may comprise the mitral valve. The prosthetic heart valve 270 may include one or more prosthetic valve leaflets 275. The ventricular chamber may form a channel from the mitral valve to the aortic valve. The ventricular chamber may direct blood flow from the atrium to the inflow area of the aortic valve 274.

The ventricular chamber may be configured to be compliant and compressed by the left ventricle during systole and expansion during diastole.

The ventricular chamber may include a portion 273 configured to be positioned in the left ventricular outflow tract (LVOT) 282. In examples, the ventricular chamber may be configured to support and maintain a patency of a left ventricular outflow tract (LVOT) 282 of the heart. In examples, other systems, apparatuses, and methods, may be utilized to reduce an obstruction of the LVOT 282.

FIG. 21 illustrates an example of a stent 280 that may be positioned in the LVOT 282 of a left ventricle. The stent 280 may be deployed proximate the aortic valve 274 and may have an outflow proximate the aortic valve 274.

The stent 280 may include an outer stent 284 for anchoring. The outer stent 284 may be configured to be deployed proximate the LVOT 282. The outer stent 284 may be self expandable and may be made of a shape memory material (such as nitinol or another form of material). The outer stent 284 may include one or more anchor arms 286 that may be configured to anchor the stent 280 in position. The anchor arms 286 may anchor the outer stent 284 proximate the LVOT 282.

The stent 280 may include an inner stent 288 that may be positioned within the outer stent 284. The inner stent 288 may comprise a balloon expandable inner stent and may include a flow channel 290 for fluid to pass through the left ventricular outflow tract 282.

The stent 280 may be configured to maintain a patency of the left ventricular outflow tract 282 during deployment of a prosthetic mitral valve 292 to the native mitral valve 294.

FIG. 22 illustrates an example in which a prosthetic aortic valve 300 may have an extended body 302 that extends into the left ventricle 276 to maintain a patency of the left ventricular outflow tract 282 during deployment of a prosthetic mitral valve 292 to the native mitral valve 294. The extended body 302 may have a length such that a mitral leaflet 303 may contact an outer surface of the extended body 302, to avoid obstructing the LVOT 282.

Other systems, apparatuses, and methods may be utilized to reduce an obstruction of the LVOT 282.

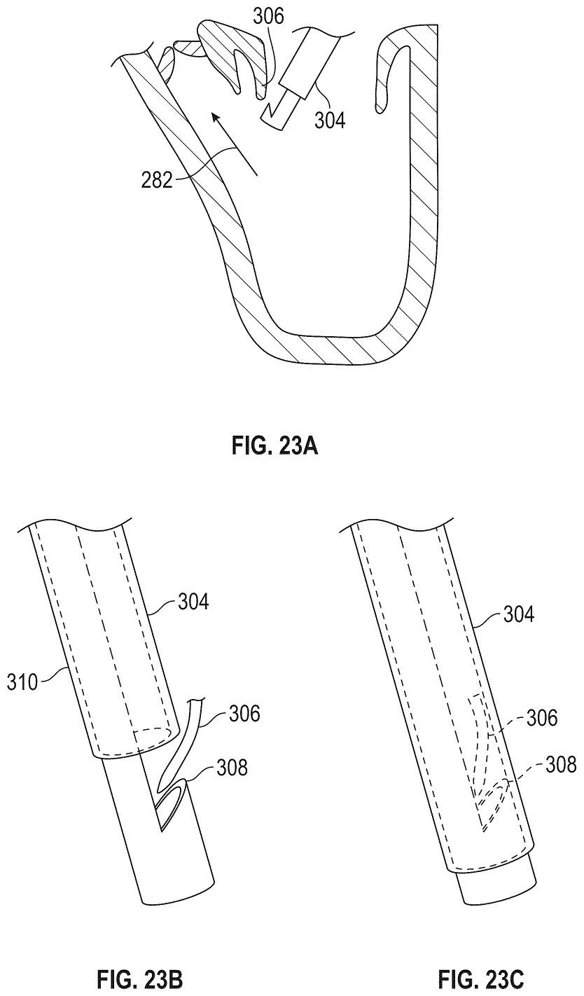

FIGS. 23A-28B illustrate exemplary methods of tethering a native mitral heart valve leaflet or removing at least a portion of the native heart valve leaflet to reduce an obstruction by the native heart valve leaflet of the left ventricular outflow tract 282 of a heart.

FIG. 23A, for example, illustrates a cutter 304 approaching the native heart valve leaflet 306. Referring to FIG. 23B, the cutter 304 may include a cutting surface 308 and a retention tube 310 that may be configured to retain all or a portion of the native heart valve leaflet 306. Referring to FIG. 23C, the cutter 304 may close upon the native heart valve leaflet 306 to cut and remove the entirety or a portion of the native heart valve leaflet 306. The native heart valve leaflet 306 may be positioned within the tube 310 in vivo.

All or a portion of the native heart valve leaflet 306 may be removed to reduce an obstruction by the native heart valve leaflet 306 of the left ventricular outflow tract 282 of a heart.

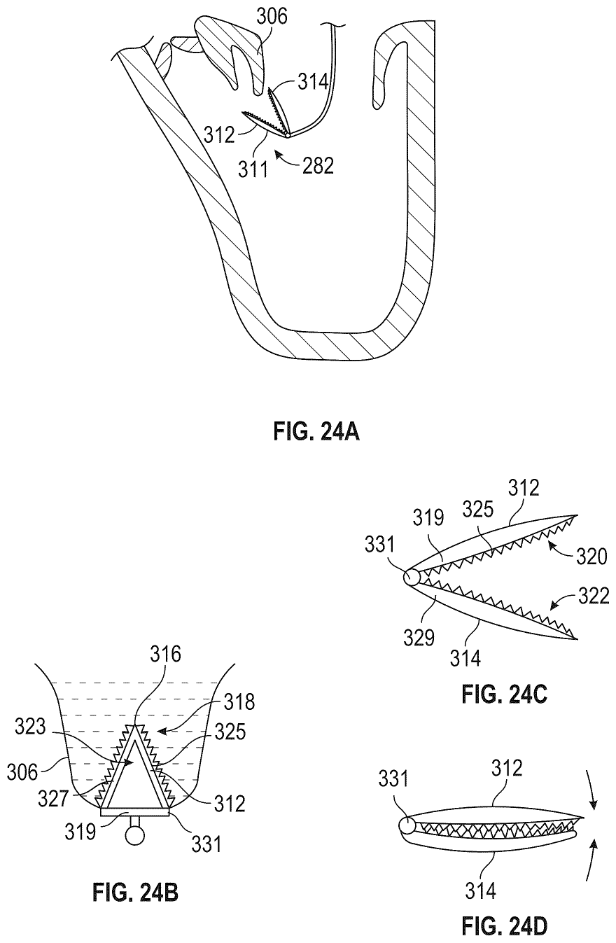

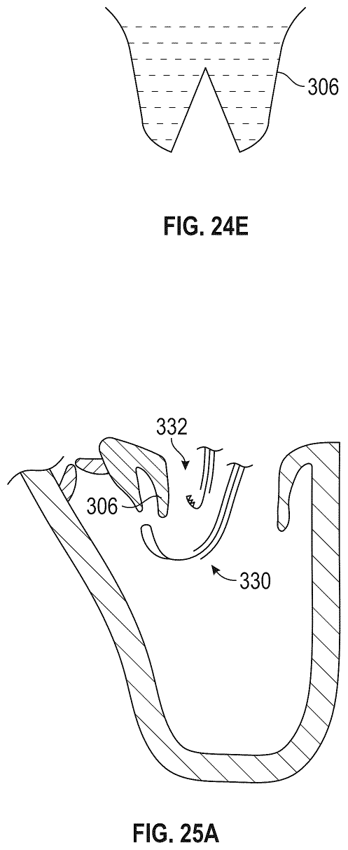

FIGS. 24A-24D illustrate an example of a cutter 311 that may be utilized in examples herein. The cutter 311 may comprise cutting jaws include a first jaw 312 and a second jaw 314. A side view of the cutter 311 is shown in FIGS. 24A, 24C, and 24D. An orthogonal view is shown in FIG. 24B.

Referring to FIG. 24B, the first jaw 312 may have a wedge shape converging on an apex 316 at a distal end portion 318 of the first jaw 312. The first jaw 312 may have a proximal end portion 319. The second jaw 314 may have a similar configuration as the first jaw 312. A pivot 331 may couple the proximal end portion 319 of the first jaw 312 to the proximal end portion of the second jaw 314, with the first jaw 312 configured to pivot about the pivot relative to the second jaw 314.

The wedge shape of the first jaw 312 and the second jaw 314 may allow the jaws 312, 314 to form a wedge-shaped cut of the native valve leaflet 306. Referring to FIG. 24C, one or more teeth 320, 322 may be positioned on one or more of the first jaw 312 or the second jaw 314 and configured to cut the at least the portion of the heart valve upon the first jaw 312 closing with the second jaw 314. The cut of the native valve leaflet 306 may be wedge shaped, as shown in FIG. 24E for example. The first jaw 312 may include a first edge 325 and an opposite edge 327 that each extend from the proximal end portion 319 to the distal end portion 318 of the first jaw 312. The second jaw 314 may include a second edge 329 and an opposite edge that each extend from the proximal end portion to the distal end portion of the second jaw 314. One or more teeth may extend along one or more of the first edge 325 or the second edge 329.

The wedge-shaped portion of the native heart valve leaflet 306 may be removed to reduce an obstruction by the native heart valve leaflet 306 of the left ventricular outflow tract 282 of the heart. For example, a space 323 (marked in FIG. 24B) between the teeth 320, 322 may retain the portion of the native heart valve leaflet 306 that is cut, to remove such portion upon removal of the cutter 311 from the heart. The space 323 may be positioned between the first edge 325 and the opposite edge 327 of the first jaw 312. A space of the second jaw 314 may be positioned between the second edge 329 and the opposite edge of the second jaw 314.

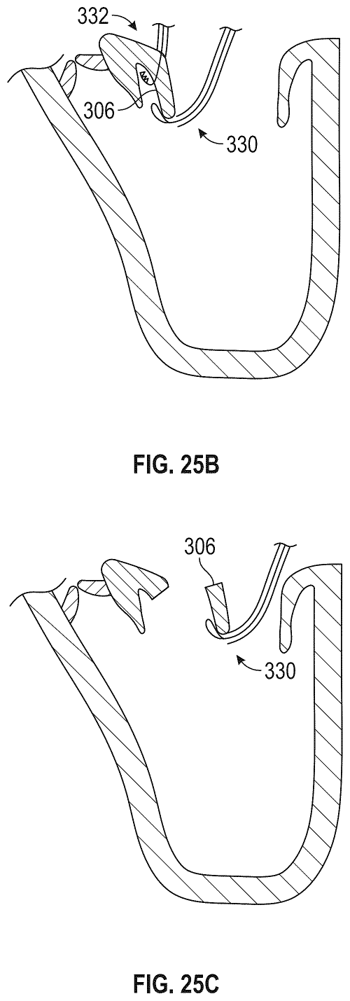

FIG. 25A illustrates an example in which a grasping snare 330 may be utilized to grasp a native heart valve leaflet 306. The grasping snare 330 may be inserted through the mitral valve annulus and may grasp the native heart valve leaflet 306. A cutter in the form of a cutting snare 332 may be utilized to cut the native heart valve leaflet 306 at a desired location.

FIG. 25B, for example, illustrates the grasping snare 330 having grasped the native heart valve leaflet 306. The grasping snare 330 may hold the leaflet 306 taut as the cutting snare 332 cuts the leaflet 306. The grasping snare 330 may continue to retain the leaflet 306 as the leaflet 306 is withdrawn from the heart.

FIG. 25C, for example, illustrates the cut leaflet 306 being withdrawn by the grasping snare 330.

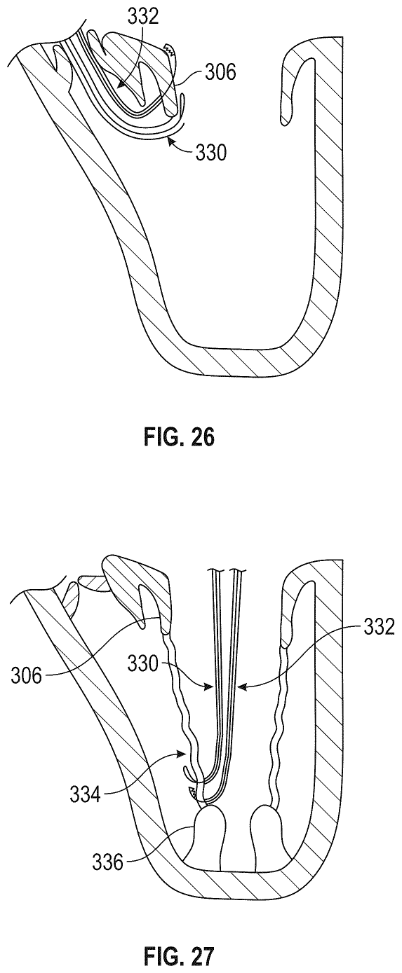

FIG. 26 illustrates an example in which the grasping snare 330 or cutting snare 332 may approach from the aortic valve. The leaflet 306 may be cut in a similar manner as the leaflet 306 shown in FIG. 25C.

Referring to FIG. 27, in examples, the chordae 334 attached to the leaflet 306 may be cut in addition to the leaflet 306. For example, a grasping snare 330 may grasp chordae 334 proximate the papillary muscles 336 of the heart. A cutting snare 332 may cut the chordae 334 between the papillary muscles 336 and the grasping snare 330. The leaflet 306 may be cut in a similar manner shown in FIGS. 25A-26.

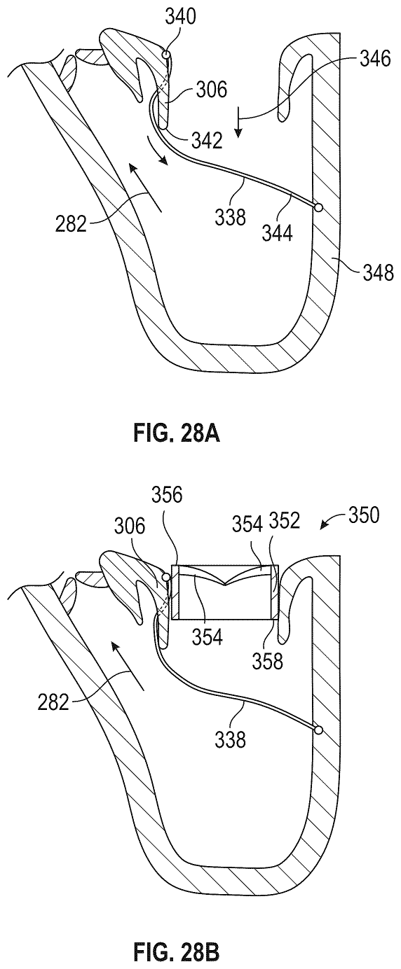

FIGS. 28A-28B illustrate an exemplary method of tethering a native mitral heart valve leaflet 306 to reduce an obstruction by the leaflet 306 of the left ventricular outflow tract 282 of the heart. A tether 338 may be configured to pass through the leaflet 306. The tether 338 may be configured to tether the native mitral heart valve leaflet 306 to reduce an obstruction by the native heart valve leaflet 306 of the left ventricular outflow tract 282 of the heart.

Referring to FIG. 28A, a first end portion 340 of the tether 338 may be positioned at a base of the leaflet 306 and may be anchored to an interior facing surface of the leaflet 306. The tether 338 may be adapted to anchor to the native mitral heart valve leaflet 306. The tether 338 may pass through the leaflet 306 to extend over an outward facing surface of the leaflet 306 and over a distal tip 342 of the leaflet 306. The tether 338, for example, may have an enlarged body at the end of the tether 338 that impedes removal of the tether 338 from the native mitral heart valve leaflet 306. The tether 338 may pass across the mitral inflow tract 346 to anchor to an opposite wall 348 of the heart. The opposite wall 348 may comprise a ventricular wall. The tether 338 may include a barb or other form of anchor to anchor to the ventricular wall.

Referring to FIG. 28B, the tether 338 may be cinched to draw the leaflet 306 away from the left ventricular outflow tract 282. As such, upon deployment of a prosthetic mitral valve 350 to the mitral valve, the possibility of obstruction of the left ventricular outflow tract 282 by the leaflet 306 may be reduced. The prosthetic mitral valve 350 may be configured similarly as any other form of prosthetic valve disclosed herein. For example, the prosthetic mitral valve 350 may include a support structure 352 that may support a valve portion as disclosed herein. For example, the support structure 352 may support one or more prosthetic valve leaflets 354 that may be positioned within a passageway of the support structure 352. The support structure 352 may have an inlet end portion 356 and an outlet end portion 358 as disclosed herein.

Variations in the systems and methods disclosed herein may be provided.

The examples of prosthetic valves may be utilized in a mitral valve as disclosed herein, or may be utilized in other deployment locations such as a native tricuspid valve, or other deployment locations unless stated otherwise. Deployment to aortic or pulmonary valves, or other implantation sites may be utilized.

Features of examples may be utilized solely, or in combination with other features disclosed herein.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present, or problems be solved. Features, elements, or combinations of one example can be combined into other examples herein.

Example 1: A system for a heart, the system comprising: a prosthetic valve configured to be deployed to a native valve of a heart and configured to transmit shock waves to break up calcification of the native valve.

Example 2: The system of any example herein, in particular example 1, wherein the prosthetic valve includes a frame configured to support one or more prosthetic valve leaflets.

Example 3: The system of any example herein, in particular example 2, wherein the frame surrounds a flow channel that the one or more prosthetic valve leaflets are positioned in.

Example 4: The system of any example herein, in particular examples 1-3, further comprising a sealing skirt and a frame that supports the sealing skirt.

Example 5: The system of any example herein, in particular examples 1-4, further comprising a frame and one or more distal anchors configured to anchor the frame to the native valve.

Example 6: The system of any example herein, in particular example 5, wherein the one or more distal anchors are each configured to extend over a tip of a leaflet of the native valve.

Example 7: The system of any example herein, in particular examples 1-6, wherein the prosthetic valve includes a frame comprising a shape memory material.

Example 8: The system of any example herein, in particular examples 1-7, wherein the prosthetic valve includes a frame comprising nitinol.

Example 9: The system of any example herein, in particular examples 1-8, wherein the prosthetic valve has a diameter, and the prosthetic valve is configured such that an electric current applied to the prosthetic valve increases the diameter.

Example 10: The system of any example herein, in particular examples 1-9, further comprising a controller configured to apply electric current to the prosthetic valve to produce the shock waves.

Example 11: The system of any example herein, in particular example 10, further comprising one or more terminals coupling the controller to the prosthetic valve to transmit the electric current to the prosthetic valve.

Example 12: The system of any example herein, in particular example 10 or example 11, wherein the controller is configured to vary an amount of the electric current applied to the prosthetic valve.

Example 13: The system of any example herein, in particular examples 10-12, wherein the controller is configured to apply pulsatile electric current to the prosthetic valve.

Example 14: The system of any example herein, in particular examples 1-13, wherein the shock waves comprise ultra sound shock waves.

Example 15: The system of any example herein, in particular examples 1-14, wherein the prosthetic valve is configured to be deployed to a mitral valve or a tricuspid valve of the heart.

Example 16: A delivery system for a heart, the delivery system comprising: a first inflatable body configured to expand a prosthetic valve positioned upon the first inflatable body to deploy the prosthetic valve to a native valve; and a second inflatable body surrounding the first inflatable body and configured to transmit shock waves to break up calcification of the native valve.

Example 17: The delivery system of any example herein, in particular example 16, further comprising an elongate shaft of a delivery apparatus, and wherein the first inflatable body and the second inflatable body are each coupled to the elongate shaft.

Example 18: The delivery system of any example herein, in particular example 17, further comprising a control mechanism for controlling a deflection of the elongate shaft.

Example 19: The delivery system of any example herein, in particular examples 16-18, wherein the first inflatable body is configured to expand the prosthetic valve positioned upon the first inflatable body and upon the second inflatable body.

Example 20: The delivery system of any example herein, in particular examples 16-19, wherein the second inflatable body has an outer surface and is configured to have the prosthetic valve slid onto the outer surface in vivo.

Example 21: The delivery system of any example herein, in particular examples 16-20, wherein: the first inflatable body includes a proximal end portion and a distal end portion; the second inflatable body includes a proximal end portion and a distal end portion; and the proximal end portion of the second inflatable body is proximal of the proximal end portion of the first inflatable body, and the distal end portion of the second inflatable body is distal of the distal end portion of the first inflatable body.

Example 22: The delivery system of any example herein, in particular examples 16-21, wherein the second inflatable body is more compliant than the first inflatable body.

Example 23: The delivery system of any example herein, in particular examples 16-22, further comprising a controller configured to control a generation of the shock waves for the second inflatable body.

Example 24: The delivery system of any example herein, in particular examples 16-23, wherein the shock waves comprise ultra sound shock waves.

Example 25: The delivery system of any example herein, in particular examples 16-24, wherein the first inflatable body is configured to deploy the prosthetic valve to a mitral valve or a tricuspid valve of the heart.

Example 26: A method comprising: reducing calcification of a native heart valve; and deploying a prosthetic heart valve to the native heart valve.

Example 27: The method of any example herein, in particular example 26, further comprising utilizing a laser or a sintering device to reduce the calcification of the native heart valve.

Example 28: The method of any example herein, in particular example 26 or example 27, further comprising reducing the calcification of the native heart valve by smoothing the calcification.

Example 29: The method of any example herein, in particular examples 26-28, further comprising reducing the calcification of the native heart valve by fracturing the calcification.

Example 30: The method of any example herein, in particular examples 26-29, further comprising applying shock waves to the calcification to reduce the calcification.

Example 31: The method of any example herein, in particular example 30, further comprising utilizing an inflatable body to apply the shock waves to the calcification.

Example 32: The method of any example herein, in particular example 30 or example 31, further comprising utilizing the prosthetic heart valve to apply the shock waves to the calcification.

Example 33: The method of any example herein, in particular examples 26-32, further comprising expanding the prosthetic heart valve in the native heart valve and applying shock waves from the prosthetic heart valve to reduce the calcification of the native heart valve.

Example 34: The method of any example herein, in particular examples 26-33, further comprising utilizing an embolic capture device to capture calcification released from the native heart valve.

Example 35: The method of any example herein, in particular examples 26-34, wherein the native heart valve comprises a mitral valve or a tricuspid valve.

Example 36: A method comprising: determining a configuration of an implantation site at a native heart valve having calcification, the implantation site being for a prosthetic heart valve; and selecting a configuration of a distal anchor of the prosthetic heart valve based on the determined configuration of the implantation site.

Example 37: The method of any example herein, in particular example 36, further comprising adjusting the distal anchor based on the determined configuration of the implantation site.

Example 38: The method of any example herein, in particular example 37, wherein the prosthetic heart valve includes a frame and the adjusting of the distal anchor comprises varying an angle of the distal anchor relative to the frame.

Example 39: The method of any example herein, in particular examples 36-38, further comprising selecting the configuration of the distal anchor of the prosthetic heart valve from a set including a plurality of different configurations of distal anchors.

Example 40: The method of any example herein, in particular examples 36-39, further comprising coupling the distal anchor to a valve body of the prosthetic heart valve.

Example 41: The method of any example herein, in particular examples 36-40, further comprising selecting the configuration of the distal anchor based on a shape of calcification at the native heart valve.

Example 42: The method of any example herein, in particular examples 36-41, further comprising selecting the configuration of the distal anchor to anchor to calcification at the native heart valve.

Example 43: The method of any example herein, in particular examples 36-42, wherein the distal anchor is configured to extend over a distal tip of a leaflet of the native heart valve.

Example 44: The method of any example herein, in particular examples 36-43, further comprising imaging the implantation site to determine the configuration of the implantation site.

Example 45: The method of any example herein, in particular examples 36-44, wherein the native heart valve comprises a mitral valve or a tricuspid valve.

Example 46: A prosthetic valve configured to be deployed to a native valve of a heart, the prosthetic valve comprising: one or more prosthetic valve leaflets configured to be positioned in a flow channel; a valve body configured to support the one or more prosthetic valve leaflets; and at least one anchor coupled to the valve body and including one or more of a barb or a pad configured to engage calcification of the native valve to anchor to the calcification.

Example 47: The prosthetic valve of any example herein, in particular example 46, wherein the valve body includes a distal end portion and a proximal end portion, and the at least one anchor comprises an arm having a first end portion coupled to the distal end portion of the valve body and a second end portion extending proximally from the first end portion of the arm.

Example 48: The prosthetic valve of any example herein, in particular example 47, wherein the second end portion is configured to be positioned interior of an inward facing surface of a heart valve leaflet upon anchoring to the calcification.

Example 49: The prosthetic valve of any example herein, in particular example 47 or example 48, wherein the second end portion is biased to extend outward from the valve body.

Example 50: The prosthetic valve of any example herein, in particular examples 47-49, wherein the second end portion is configured to be positioned radially outward of an inward facing surface of a heart valve leaflet upon anchoring to the calcification.

Example 51: A method comprising: deploying a prosthetic valve to a native valve, the prosthetic valve comprising: one or more prosthetic valve leaflets configured to be positioned in a flow channel, a valve body configured to support the one or more prosthetic valve leaflets, and at least one anchor coupled to the valve body and including one or more of a barb or a pad configured to engage calcification of the native valve to anchor to the calcification.

Example 52: The method of any example herein, in particular example 51, wherein the valve body includes a distal end portion and a proximal end portion, and the at least one anchor comprises an arm having a first end portion coupled to the distal end portion of the valve body and a second end portion extending proximally from the first end portion of the arm.

Example 53: The method of any example herein, in particular example 52, wherein the second end portion is configured to be positioned interior of an inward facing surface of a heart valve leaflet upon anchoring to the calcification.

Example 54: The method of any example herein, in particular example 52 or example 53, wherein the second end portion is biased to extend outward from the valve body.

Example 55: The method of any example herein, in particular examples 52-54, wherein the second end portion is configured to be positioned radially outward of an inward facing surface of a heart valve leaflet upon anchoring to the calcification.

Example 56: A prosthetic valve configured to be deployed to a native valve of a heart, the prosthetic valve comprising: one or more prosthetic valve leaflets configured to be positioned in a flow channel; and a valve body configured to support the one or more prosthetic valve leaflets and including an outer surface comprising a conformable anchoring surface configured to conform to a shape of calcification of the native valve to anchor to the native valve.

Example 57: The prosthetic valve of any example herein, in particular example 56, wherein the conformable anchoring surface comprises a surface of a chamber configured to retain a fluid.

Example 58: The prosthetic valve of any example herein, in particular example 57, wherein the fluid comprises a hydrogel.

Example 59: The prosthetic valve of any example herein, in particular examples 56-58, wherein the conformable anchoring surface is configured to deflect radially inward to conform to the shape of the calcification.

Example 60: The prosthetic valve of any example herein, in particular examples 56-59, wherein the valve body includes a frame positioned radially inward of the conformable anchoring surface.

Example 61: A method comprising: deploying a prosthetic valve to a native valve, the prosthetic valve comprising: one or more prosthetic valve leaflets configured to be positioned in a flow channel, and a valve body configured to support the one or more prosthetic valve leaflets and including an outer surface comprising a conformable anchoring surface configured to conform to a shape of calcification of the native valve to anchor to the native valve.

Example 62: The method of any example herein, in particular example 61, wherein the conformable anchoring surface comprises a surface of a chamber configured to retain a fluid.

Example 63: The method of any example herein, in particular example 62, wherein the fluid comprises a hydrogel.

Example 64: The method of any example herein, in particular examples 61-63, wherein the conformable anchoring surface is configured to deflect radially inward to conform to the shape of the calcification.

Example 65: The method of any example herein, in particular examples 61-64, wherein the valve body includes a frame positioned radially inward of the conformable anchoring surface.

Example 66: A prosthetic valve configured to be deployed to a native valve, the prosthetic valve comprising: one or more prosthetic valve leaflets configured to be positioned in a flow channel; and a valve body configured to support the one or more prosthetic valve leaflets, the valve body including an atrial anchor comprising a flange configured to extend radially outward from the flow channel.

Example 67: The prosthetic valve of any example herein, in particular example 66, wherein the flange comprises an inflatable body.

Example 68: The prosthetic valve of any example herein, in particular example 67, wherein the inflatable body comprises a ring extending around the valve body.

Example 69: The prosthetic valve of any example herein, in particular examples 66-68, wherein the valve body includes a plurality of barbs extending radially outward from the flow channel.

Example 70: The prosthetic valve of any example herein, in particular examples 66-69, further comprising an anchor configured to anchor to a ventricular wall and a tether configured to couple the anchor to the valve body.

Example 71: The prosthetic valve of any example herein, in particular example 70, wherein the tether comprises a compliant tether configured to resist a force in an atrial direction applied to the valve body.

Example 72: The prosthetic valve of any example herein, in particular examples 66-71, wherein the one or more prosthetic valve leaflets are positioned proximal of the atrial anchor.

Example 73: The prosthetic valve of any example herein, in particular examples 66-72, wherein the valve body is positioned proximal of the atrial anchor.

Example 74: The prosthetic valve of any example herein, in particular examples 66-73, further comprising one or more penetrating bodies configured to pass through the flange to anchor the valve body to the native valve.

Example 75: The prosthetic valve of any example herein, in particular examples 66-74, wherein the prosthetic valve is configured to be deployed to a mitral valve or a tricuspid valve of a heart.

Example 76: A method comprising: deploying a prosthetic valve to a native valve, the prosthetic valve comprising: one or more prosthetic valve leaflets configured to be positioned in a flow channel, and a valve body configured to support the one or more prosthetic valve leaflets, the valve body including an atrial anchor comprising a flange configured to extend radially outward from the flow channel.

Example 77: The method of any example herein, in particular example 76, wherein the flange comprises an inflatable body.

Example 78: The method of any example herein, in particular example 77, wherein the inflatable body comprises a ring extending around the valve body.

Example 79: The method of any example herein, in particular examples 76-78, wherein the valve body includes a plurality of barbs extending radially outward from the flow channel.

Example 80: The method of any example herein, in particular examples 76-79, further comprising an anchor configured to anchor to a ventricular wall and a tether configured to couple the anchor to the valve body.

Example 81: The method of any example herein, in particular example 80, wherein the tether comprises a compliant tether configured to resist a force in an atrial direction applied to the valve body.

Example 82: The method of any example herein, in particular examples 76-81, wherein the one or more prosthetic valve leaflets are positioned proximal of the atrial anchor.

Example 83: The method of any example herein, in particular examples 76-82, wherein the valve body is positioned proximal of the atrial anchor.

Example 84: The method of any example herein, in particular examples 76-83, further comprising one or more penetrating bodies configured to pass through the flange to anchor the valve body to the native valve.

Example 85: The method of any example herein, in particular examples 76-84, wherein the prosthetic valve is deployed to a mitral valve or a tricuspid valve of a heart.

Example 86: A prosthetic valve configured to be deployed to a native valve, the prosthetic valve comprising: a valve body; and a spiral body coupled to the valve body and configured to move between an opened state and a closed state to control fluid flow through the valve body.

Example 87: The prosthetic valve of any example herein, in particular example 86, wherein the spiral body is configured to move distally to move from the closed state to the opened state.

Example 88: The prosthetic valve of any example herein, in particular example 86 or example 87, wherein the spiral body includes an arm forming a spiral and having a radially inward portion and a radially outward portion, and the radially inward portion is coplanar with the radially outward portion in the closed state.

Example 89: The prosthetic valve of any example herein, in particular example 88, wherein the radially inward portion is distal of the radially outward portion in the opened state.

Example 90: The prosthetic valve of any example herein, in particular example 88 or example 89, wherein one or more gaps between the radially inward portion and the radially outward portion are formed when the spiral body moves to the opened state, and the one or more gaps are closed when the spiral body is in the closed state.

Example 91: The prosthetic valve of any example herein, in particular examples 86-90, wherein the spiral body is configured to cyclically move between the opened state and the closed state.

Example 92: The prosthetic valve of any example herein, in particular examples 86-91, wherein a force of fluid is configured to move the spiral body between the opened state and the closed state.

Example 93: The prosthetic valve of any example herein, in particular examples 86-92, wherein the valve body comprises a ring extending around the spiral body.

Example 94: The prosthetic valve of any example herein, in particular examples 86-93, further comprising one or more penetrating bodies configured to pass through the valve body to anchor the valve body to the native valve.

Example 95: The prosthetic valve of any example herein, in particular examples 86-94, wherein the prosthetic valve is configured to be deployed to a mitral valve or a tricuspid valve of a heart.

Example 96: A method comprising: deploying a prosthetic valve to a native valve, the prosthetic valve comprising: a valve body, and a spiral body coupled to the valve body and configured to move between an opened state and a closed state to control fluid flow through the valve body.

Example 97: The method of any example herein, in particular example 96, wherein the spiral body is configured to move distally to move from the closed state to the opened state.

Example 98: The method of any example herein, in particular example 96 or example 97, wherein the spiral body includes an arm forming a spiral and having a radially inward portion and a radially outward portion, and the radially inward portion is coplanar with the radially outward portion in the closed state.

Example 99: The method of any example herein, in particular example 98, wherein the radially inward portion is distal of the radially outward portion in the opened state.

Example 100: The method of any example herein, in particular example 98 or example

99, wherein one or more gaps between the radially inward portion and the radially outward portion are formed when the spiral body moves to the opened state, and the one or more gaps are closed when the spiral body is in the closed state.

Example 101: The method of any example herein, in particular examples 96-100, wherein the spiral body is configured to cyclically move between the opened state and the closed state.

Example 102: The method of any example herein, in particular examples 96-101, wherein a force of fluid is configured to move the spiral body between the opened state and the closed state.

Example 103: The method of any example herein, in particular examples 96-102, wherein the valve body comprises a ring extending around the spiral body.

Example 104: The method of any example herein, in particular examples 96-103, further comprising one or more penetrating bodies configured to pass through the valve body to anchor the valve body to the native valve.

Example 105: The method of any example herein, in particular examples 96-104, wherein the prosthetic valve is deployed to a mitral valve or a tricuspid valve of a heart.

Example 106: A system for a heart, the system comprising: a prosthetic heart valve configured to be deployed in a mitral valve of the heart; and an anchor coupled to the prosthetic heart valve and configured to be deployed in a left atrial appendage of the heart.

Example 107: The system of any example herein, in particular example 106, wherein a tether couples the prosthetic heart valve to the anchor and is configured to extend within a left atrium of the heart.

Example 108: The system of any example herein, in particular example 106 or example 107, wherein the anchor comprises a stent.

Example 109: The system of any example herein, in particular examples 106-108, wherein the prosthetic heart valve includes one or more prosthetic valve leaflets coupled to a frame.

Example 110: The system of any example herein, in particular examples 106-109, wherein the anchor is configured to retain the prosthetic heart valve in position in the mitral valve of the heart.

Example 111: A method comprising: deploying a prosthetic heart valve to a mitral valve of a heart; and deploying an anchor for the prosthetic heart valve to a left atrial appendage of the heart.

Example 112: The method of any example herein, in particular example 111, wherein a tether couples the prosthetic heart valve to the anchor and is configured to extend within a left atrium of the heart.

Example 113: The method of any example herein, in particular example 111 or example 112, wherein the anchor comprises a stent.

Example 114: The method of any example herein, in particular examples 111-113, wherein the prosthetic heart valve includes one or more prosthetic valve leaflets coupled to a frame.

Example 115: The method of any example herein, in particular examples 111-114, wherein the anchor is configured to retain the prosthetic heart valve in position in the mitral valve of the heart.

Example 116: A method comprising: implanting a prosthetic valve at a pulmonary vein of a heart to impede fluid flow to a lung.

Example 117: The method of any example herein, in particular example 116, wherein the prosthetic valve includes a frame coupled to one or more prosthetic valve leaflets.

Example 118: The method of any example herein, in particular example 116 or example 117, wherein the prosthetic valve is configured to open in a flow direction towards a left atrium of the heart.

Example 119: The method of any example herein, in particular examples 116-118, further comprising implanting a plurality of prosthetic valves into a plurality of pulmonary veins of the heart.

Example 120: The method of any example herein, in particular examples 116-119, wherein a mitral valve of the heart includes calcification.

Example 121: A stent for a heart, the stent comprising: an outer stent configured to be deployed proximate a left ventricular outflow tract of the heart; and an inner stent positioned within the outer stent and including a flow channel for fluid to pass through the left ventricular outflow tract.

Example 122: The stent of any example herein, in particular example 121, wherein the inner stent is balloon expandable.

Example 123: The stent of any example herein, in particular example 121 or example 122, wherein the outer stent is self expandable.

Example 124: The stent of any example herein, in particular examples 121-123, wherein the outer stent is made of a shape memory material.

Example 125: The stent of any example herein, in particular examples 121-124, wherein the outer stent includes one or more anchor arms for anchoring the outer stent proximate the left ventricular outflow tract.

Example 126: A method comprising: deploying a stent proximate a left ventricular outflow tract of a heart, the stent including: an outer stent, and an inner stent positioned within the outer stent and including a flow channel for fluid to pass through the left ventricular outflow tract.

Example 127: The method of any example herein, in particular example 126, wherein the inner stent is balloon expandable.

Example 128: The method of any example herein, in particular example 126 or example 127, wherein the outer stent is self expandable.

Example 129: The method of any example herein, in particular examples 126-128, wherein the outer stent is made of a shape memory material.

Example 130: The method of any example herein, in particular examples 126-129, wherein the outer stent includes one or more anchor arms for anchoring the outer stent proximate the left ventricular outflow tract.

Example 131: A system for a heart, the system comprising: a prosthetic heart valve configured to be implanted in a valve of the heart; and an anchor coupled to the prosthetic heart valve and comprising a ventricular chamber configured to extend with a ventricle of the heart.

Example 132: The system of any example herein, in particular example 131, wherein the ventricular chamber is configured to be compressed by the ventricle of the heart.

Example 133: The system of any example herein, in particular example 131 or example 132, wherein the prosthetic heart valve is configured to be positioned in a mitral valve of the heart and the ventricular chamber is configured to form a channel from the mitral valve to an aortic valve of the heart.

Example 134: The system of any example herein, in particular examples 131-133, wherein the ventricular chamber includes a portion configured to be positioned within a left ventricular outflow tract of the heart.

Example 135: The system of any example herein, in particular examples 131-134, wherein the prosthetic heart valve includes one or more prosthetic valve leaflets.

Example 136: A method comprising: deploying a prosthetic heart valve to a valve of a heart; and deploying an anchor for the prosthetic heart valve to a ventricle of the heart, the anchor comprising a ventricular chamber.

Example 137: The method of any example herein, in particular example 136, wherein the ventricular chamber is configured to be compressed by the ventricle of the heart.

Example 138: The method of any example herein, in particular example 136 or example 137, wherein the prosthetic heart valve is configured to be positioned in a mitral valve of the heart and the ventricular chamber is configured to form a channel from the mitral valve to an aortic valve of the heart.

Example 139: The method of any example herein, in particular examples 136-138, wherein the ventricular chamber includes a portion configured to be positioned within a left ventricular outflow tract of the heart.

Example 140: The method of any example herein, in particular examples 136-139, wherein the prosthetic heart valve includes one or more prosthetic valve leaflets.

Example 141: A method comprising: tethering a native mitral heart valve leaflet or removing at least a portion of the native mitral heart valve leaflet to reduce an obstruction by the native mitral heart valve leaflet of a left ventricular outflow tract of a heart.

Example 142: The method of any example herein, in particular example 141, further comprising removing an entirety of the native mitral heart valve leaflet.

Example 143: The method of any example herein, in particular example 141 or example 142, further comprising removing a wedge shaped portion of the native mitral heart valve leaflet.

Example 144: The method of any example herein, in particular examples 141-143, further comprising utilizing a cutting snare to remove at least the portion of the native mitral heart valve leaflet.

Example 145: The method of any example herein, in particular examples 141-144, further comprising utilizing a cutting snare to cut chordae attached to the native mitral heart valve leaflet.

Example 146: The method of any example herein, in particular examples 141-145, further comprising utilizing cutting jaws to remove at least the portion of the native mitral heart valve leaflet.

Example 147: The method of any example herein, in particular examples 141-146, further comprising positioning the at least the portion of the native mitral heart valve leaflet within a tube in vivo.

Example 148: The method of any example herein, in particular examples 141-147, further comprising: passing a tether through the native mitral heart valve leaflet; and anchoring the tether to a ventricular wall to tether the native mitral heart valve leaflet.

Example 149: The method of any example herein, in particular examples 141-148, further comprising deploying a prosthetic heart valve to a mitral valve that includes the native mitral heart valve leaflet.

Example 150: The method of any example herein, in particular examples 141-149, wherein a mitral valve that includes the native mitral heart valve leaflet has calcification.

Example 151: A cutter for at least a portion of a heart valve leaflet, the cutter comprising: a first jaw including a proximal end portion and a distal end portion, the first jaw having a wedge shape converging on an apex at the distal end portion of the first jaw; a second jaw including a proximal end portion and a distal end portion, the second jaw having a wedge shape converging on an apex at the distal end portion of the second jaw; and one or more teeth positioned on one or more of the first jaw or the second jaw and configured to cut the at least the portion of the heart valve leaflet upon the first jaw closing with the second jaw.

Example 152: The cutter of any example herein, in particular example 151, wherein the first jaw includes a first edge extending from the proximal end portion to the distal end portion of the first jaw, and the second jaw includes a second edge extending from the proximal end portion to the distal end portion of the second jaw, and the one or more teeth extend along one or more of the first edge or the second edge.

Example 153: The cutter of any example herein, in particular example 152, wherein the one or more teeth are positioned on the first edge and on the second edge.

Example 154: The cutter of any example herein, in particular example 152 or example 153, wherein the first jaw includes a third edge extending from the proximal end portion to the distal end portion of the first jaw opposite the first edge, and the at least the portion of the heart valve leaflet is configured to be retained between the first edge and the third edge.

Example 155: The cutter of any example herein, in particular examples 151-154, further comprising a pivot coupling the proximal end portion of the first jaw to the proximal end portion of the second jaw, the first jaw configured to pivot about the pivot relative to the second jaw.

Any of the features of any of the examples, including but not limited to any of the first through 155 examples referred to above, is applicable to all other aspects and examples identified herein, including but not limited to any examples of any of the first through 155 examples referred to above. Moreover, any of the features of an example of the various examples, including but not limited to any examples of any of the first through 155 examples referred to above, is independently combinable, partly, or wholly with other examples described herein in any way, e.g., one, two, or three or more examples may be combinable in whole or in part. Further, any of the features of the various examples, including but not limited to any examples of any of the first through 155 examples referred to above, may be made optional to other examples. Any example of a method can be performed by a system or apparatus of another example, and any aspect or example of a system or apparatus can be configured to perform a method of another aspect or example, including but not limited to any examples of any of the first through 155 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system for implanting a prosthetic heart valve in a calcified native valve, the system comprising:
the prosthetic heart valve including:
a support structure having an inlet end portion and an outlet end portion and a passageway, and
a valve portion positioned within the passageway of the support structure, wherein the valve portion comprises a plurality of leaflets made from pericardium, wherein the valve portion permits flow of blood through the passageway in one direction for replacing the function of a native heart valve; and
a delivery catheter for delivering the prosthetic heart valve to the calcified native valve, the delivery catheter including an actuation mechanism for causing the support structure to vibrate, thereby reducing calcification of the calcified native valve.

2. The system of claim 1, wherein the support structure includes an actuator for producing vibrations of the support structure, to thereby reduce the calcification of the calcified native valve.

3. The system of claim 2, wherein the actuator includes electrodes for vaporizing fluid within an inflatable body positioned upon the support structure, or a piezoelectric actuator.

4. The system of claim 2, wherein the actuation mechanism includes one or more electric terminals for electrical conduction with one or more electric terminals of the actuator.

5. The system of claim 1, wherein the support structure includes a support frame, and the actuation mechanism is adapted to apply vibrations to the support frame to cause the support structure to vibrate, thereby reducing the calcification of the calcified native valve.

6. The system of claim 5, wherein the actuation mechanism includes electrodes for vaporizing fluid within an inflatable body applied to the support frame to apply the vibrations to the support frame, or a piezoelectric actuator adapted to apply the vibrations to the support frame.

7. The system of claim 1, wherein the support structure includes a support frame made of a shape memory material, and the actuation mechanism includes one or more electric terminals for applying electrical energy to the support frame to cause the support frame to expand.

8. The system of claim 1, wherein the actuation mechanism includes a controller for controlling energy that is transmitted to the support structure to cause the support structure to vibrate, and the system further comprises one or more sensors for providing feedback to the controller of a diameter of the prosthetic heart valve.

9. The system of claim 1, wherein the actuation mechanism is configured to cause the support structure to produce ultrasonic waves.

10. A prosthetic mitral heart valve system for a heart, the system comprising:
a prosthetic mitral heart valve;
an anchor for deployment in a left atrial appendage of the heart, wherein the anchor is coupled to the prosthetic mitral heart valve for anchoring the prosthetic mitral heart valve within a native mitral heart valve.

11. The system of claim 10, wherein a tether couples the prosthetic mitral heart valve to the anchor and is adapted to extend within a left atrium of the heart.

12. The system of claim 10, wherein the anchor comprises a stent.

13. The system of claim 10, wherein the prosthetic mitral heart valve comprises:
a support structure having a passageway; and
a spiral body coupled to the support structure and positioned within the passageway, the spiral body adapted to move between an opened state and a closed state to control blood flow through the support structure.

14. The system of claim 13, wherein the spiral body includes an arm forming a spiral and having a radially inward portion and a radially outward portion, and the radially inward portion is coplanar with the radially outward portion in the closed state, and
wherein one or more gaps between the radially inward portion and the radially outward portion are formed when the spiral body moves to the opened state, and the one or more gaps are closed when the spiral body is in the closed state.

15. The system of claim 10, wherein the prosthetic mitral heart valve comprises:
a support structure having an inlet end portion and an outlet end portion and a passageway, wherein the support structure includes an atrial anchor comprising a flange for extending radially outward from the passageway; and
a valve portion positioned within the passageway of the support structure, wherein the valve portion comprises a plurality of leaflets made from pericardium, wherein the valve portion permits flow of blood through the passageway in one direction for replacing the function of the native mitral heart valve.

16. The system of claim 15, wherein the flange comprises an inflatable body, and the inflatable body comprises a ring extending around the support structure.

17. A cutter for at least a portion of a heart valve leaflet, the cutter comprising:
a first jaw including a proximal end portion and a distal end portion, the first jaw having a wedge shape converging on an apex at the distal end portion of the first jaw;
a second jaw including a proximal end portion and a distal end portion, the second jaw having a wedge shape converging on an apex at the distal end portion of the second jaw; and
one or more teeth positioned on one or more of the first jaw or the second jaw and configured to cut the at least the portion of the heart valve leaflet upon the first jaw closing with the second jaw.

18. The cutter of claim 17, wherein the first jaw includes a first edge extending from the proximal end portion to the distal end portion of the first jaw, and the second jaw includes a second edge extending from the proximal end portion to the distal end portion of the second jaw, and the one or more teeth extend along one or more of the first edge or the second edge.

19. The cutter of claim 18, wherein the one or more teeth are positioned on the first edge and on the second edge.

20. A prosthetic mitral heart valve system for a heart, the system comprising:
a prosthetic mitral heart valve including:
a support structure having an inlet end portion and an outlet end portion and a passageway, and
a valve portion positioned within the passageway of the support structure, wherein the valve portion comprises a plurality of leaflets made from pericardium, wherein the valve portion permits flow of blood through the passageway in one direction for replacing the function of a native mitral heart valve; and
a tether for tethering a native mitral heart valve leaflet to reduce an obstruction by the native mitral heart valve leaflet of a left ventricular outflow tract of the heart.
